(19) United States
(12) Patent Application Publication
(10) Pub. No.: US 2012/0070836 A1
(43) Pub. Date: Mar. 22, 2012

(54) METHODS OF DETECTING RESIDUAL AMOUNTS OF POLYMERS USED IN THE PURIFICATION OF BIOMOLECULES

(75) Inventors: Martin Zillmann, Shewsbury, MA (US); Jad Jaber, Sudbury, MA (US); James Hamzik, Chelmsford, MA (US)

(73) Assignee: Millipore Corporation, Billerica, MA (US)

(21) Appl. No.: 13/155,912

(22) Filed: Jun. 8, 2011

Related U.S. Application Data
(60) Provisional application No. 61/397,186, filed on Jun. 8, 2010.

(51) Int. Cl. C12Q 1/68; G01N 21/64; G01N 33/53; C12Q 1/02

(52) U.S. Cl. 435/6.12; 435/29; 435/7.1; 435/7.5; 435/7.92; 436/501; 435/6.1; 436/94

(57) ABSTRACT

The present invention relates to methods of detecting residual amounts of polymers including stimulus responsive polymers, which are used in processes for purifying biomolecules such as, for example, proteins, polypeptides, antibodies, vaccines and the like.

### PRIORITY

The present application claims she benefit of priority of U.S. Provisional Patent Application No. 61/397,186, filed on Jun. 8, 2010, the entire content of which is incorporated by reference herein in its entirety.

### FIELD OF THE INVENTION

The present invention relates to methods of detecting residual amounts of polymers which are used in processes for purifying biomolecules such as, for example, proteins, polypeptides, antibodies, vaccines and the like.

### BACKGROUND

Efficient and economic large scale purification of biomolecules such as, e.g., therapeutic proteins including antibodies, is an increasingly important consideration for the biotechnology and pharmaceutical industries. Generally, the purification processes are quite elaborate and expensive and include many different steps. For example, typically, in the case of proteins, proteins are produced using cell culture methods, e.g., using either mammalian or bacterial cell lines engineered to produce the protein of interest by insertion of a recombinant plasmid containing the gene encoding that protein. In general, following the expression of the target protein, its separation from one or more undesired components including, e.g., host cell proteins, media by-products and DNA, poses a formidable challenge. Such separation is especially important when the therapeutic proteins are meant for use in humans and have to be approved by the Food and Drug Administration (FDA).

In general, separation and/or purification processes that are currently being used for proteins, include at least the following steps: cell lysis to recover an intracellular protein or recovery of a protein from the media in case of a secreted protein; removal of cells and cellular debris using differential centrifugation or filtration to obtain a clarified sample containing the protein of interest; and use of a variety of chromatography media in a multi-step process to separate a protein of interest from the various impurities in the sample.

It has been demonstrated that certain polymers are especially useful in the purification of biomolecules from one or more impurities in a sample. For example, the use of polyelectrolyte polymers in flocculation to purify proteins is well established (see, e.g., International PCT Patent Application No. WO2008/091740). This can be accomplished with a wide range of polymers, with the only required general characteristic being the polymer must have some level of interaction with a species of interest (e.g., a target molecule or an impurity). Further, U.S. Patent Publication Nos., 20080255027, 20090036651, 20090232737 and 20110020327, each of which are incorporated by reference herein in their entirety, discuss the use of certain polymers, referred to as smart polymers, which are soluble in an aqueous based solvent under a certain set of process conditions such as pH, temperature and/or salt concentration and are rendered insoluble upon a change in one or more of such conditions and subsequently precipitate out.

In the case of smart polymers, it has been found that these polymers may either bind one or more soluble and/or insoluble impurities or they may bind the biomolecule of interest (e.g., the target protein being isolated). In general, such polymers can be precipitated out of solution following exposure to a stimulus (e.g., pH, temperature, salt etc.), as described in one or more of 20080255027, 20090036651, 20090232737 and 20110020327, incorporated by reference herein in their entirety.

Although, the polymers can be precipitated out of solution, the precipitation step does not always remove all of the polymer present in the sample or the biological material containing stream, thereby resulting in the presence of residual amounts of the polymer in a sample containing the biomolecule of interest. Detection of residual amounts of polymer is especially crucial when the biomolecule of interest is a therapeutic protein, e.g., when the protein is meant for use in humans and requires approval by the Food and Drug Administration (FDA).

### SUMMARY OF THE INVENTION

The present invention provides a novel and a more sensitive method, relative to prior art methods, of detecting residual amounts of a polymer used for enriching a biomolecule of interest in a sample. Furthermore, the methods of the present invention do not require sample processing and can be carried out using equipment commonly found in laboratories, e.g., a polymerase chain reaction (PCR) thermocycler machine in case of oligonucleotide tag based methods described herein.

In one aspect according to the present invention, a method of detecting residual amounts of a polymer in a sample comprising a biomolecule of interest is provided, where the polymer is used for separating the biomolecule of interest from one or more impurities. The polymer may either bind one or more impurities or it may bind the biomolecule of interest, thereby to separate the biomolecule of interest from one or more impurities. In some embodiments, the polymer may bind both the biomolecule of interest as well as one or more impurities, where the biomolecule of interest is subsequently selectively eluted, whereas the one or more impurities remain bound to the polymer.

In some embodiments, a method for detecting residual amounts of a polymer according to the claimed invention comprises the steps of: (1) contacting a sample containing the biomolecule of interest and one or more impurities with a polymer, where the polymer is associated with a tag; and (2) detecting the tag, where the amount of tag detected is indicative of the amount of residual polymer in the sample comprising the biomolecule of interest.

In some embodiments, the tag is an oligonucleotide molecule. In other embodiments, the tag is a non-oligonucleotide, molecule, e.g., a hapten molecule, a fluorescent molecule or a radioactive molecule. In instances, where a molecule is the detectable moiety, the terms “tag” and “molecule” may be used interchangeably herein.

A tag may either be attached to a polymer being detected or it may be attached to an oligonucleotide molecule which is in turn attached to the polymer being detected. Alternatively, the tag may be attached to a probe which hybridizes to the oligonucleotide molecule which is attached to the polymer being detected.

In case of an oligonucleotide molecule, the molecule may be detected using amplification means (e.g., PCR) or non-amplification means (e.g., use of a probe which hybridizes to the oligonucleotide molecule or a fluorescent molecule, hapten molecule or radioactive molecule attached to the oligonucleotide molecule or attached to a probe which hybridizes to the oligonucleotide molecule).

In some embodiments, a polymer is a stimulus responsive polymer. In sonic embodiments, the polymer is used for clarification (i.e., binding to one or more impurities in a sample containing a biomolecule of interest and one or more impurities). In other embodiments, the polymer is used for capture (i.e., binding to the biomolecule of interest). In still other embodiments, a polymer binds to both the biomolecule of interest and one or more impurities, where the biomolecule of interest is subsequently selectively eluted from the polymer, whereas the one or more impurities remain bound to the polymer.

Accordingly, in some embodiments, a method for detecting residual amounts of a stimulus responsive polymer is provided, where the method comprises the steps of: (1) contacting a solution containing a biomolecule of interest and one more impurities with a stimulus responsive polymer, where the polymer is associated with a tag, such that to form a complex of polymer and one or more impurities; (2) applying a stimulus to the solution, thereby to precipitate the complex; (3) removing the precipitate from the solution; and (4) detecting the tag in the solution containing the biomolecule of interest, where the amount of tag detected is indicative of the amount of residual polymer in the solution comprising the biomolecule of interest.

In another embodiments, a method for detecting residual amounts of a stimulus responsive polymer comprises the steps of: (1) contacting a solution containing a biomolecule of interest and one or more impurities with a stimulus responsive polymer, where the polymer is associated with a tag and where the polymer forms complexes with both the biomolecule of interest as well as the one or more impurities under a first set of conditions; (2) adding a stimulus to the solution, thereby to precipitate the complexes; (3) subjecting the precipitate to a second set of conditions, thereby to selectively elute the biomolecule of interest from the complex; and (4) detecting the tag in the eluate containing the biomolecule of interest, where the amount of tag detected is indicative of the amount of residual polymer in the eluate.

In some embodiments, there is a wash step between steps (2) and (3).

In some embodiments, the stimulus responsive polymer is responsive to a salt stimulus or a pH stimulus. In a particular embodiment, the polymer is responsive to a multivalent anion stimulus.

Exemplary stimulus responsive polymers include, but are not limited to, polyvinylamine, polyallylamine, polyvinylpyridine and polymers modified with a hydrophobic group.

In various embodiments, the tag (e.g., an oligonucleotide molecule or a non-oligonucleotide molecule such as, a hapten molecule, a fluorescent molecule or a radioactive molecule) is covalently attached to the polymer, or the tag (e.g., a hapten molecule, a fluorescent molecule or a radioactive molecule) is covalently attached to the oligonucleotide which in turn is attached to the polymer. In some embodiments, the tag is attached to a probe which hybridizes to the oligonucleotide molecule which in turn is attached to the polymer.

In some embodiments, where the tag used is an oligonucleotide molecule, the detection step comprises an amplification reaction. In some embodiments, the amplification reaction comprises use of a set of primers which hybridize to the 5′ and the 3′ ends of the oligonucleotide molecule. In a particular embodiment, the amplification reaction comprises use of a polymerase chain reaction (PCR).

In sonic embodiments, where the tag used is an oligonucleotide molecule the detection step comprises a non-amplification reaction. In an exemplary embodiment, the non-amplification reaction comprises use of a labeled probe which hybridizes to the oligonucleotide molecule. In a particular embodiment, the labeled probe comprises a detectable dye. In another exemplary embodiment, the non-amplification reaction comprises the use of a hapten tag which is attached to an oligonucleotide molecule. In yet another exemplary embodiment, the non-amplification reaction comprises the use of a radioactive tag or a fluorescent tag attached to the oligonucleotide molecule. In still other embodiments, a hapten, fluorescent or radioactive tag is attached to a probe which hybridizes to the oligonucleotide molecule attached to a polymer, which is being detected.

In some embodiments, an oligonucleotide molecule used in the methods of the present invention comprises a length which is 60 nucleotides or shorter. In some embodiments, the length of the oligonucleotide tag is selected from the group consisting of 10 nucleotides, 15, nucleotides, 20 nucleotides, 25 nucleotides, 30 nucleotides, 35 nucleotides, 40 nucleotides, 45 nucleotides, 50 nucleotides, 55 nucleotides and 60 nucleotides.

In some embodiments, the oligonucleotide molecule comprises a reactive group at the 3′ end of the tag. The reactive group may be chosen from a hydroxyl group, an amino group, a halogen group, an epoxy group, a carboxyl group, and a sulfhydryl group. In some embodiments, the reactive group may be further modified, e.g., to create yet another reactive group which interacts with the reactive group on the polymer.

In some embodiments, the polymer comprises a reactive group. The reactive group may be chosen from an aldehyde group, an epoxy group, a carboxylic acid group, a hydroxyl group, an amino group (primary, secondary, or tertiary, including pyridine), a sulfhydryl group, and an aromatic group.

In still other embodiments, the oligonucleotide molecule comprises a first reactive group and the polymer comprises a second reactive group, where the first and second reactive groups are covalently attached to each other. The reactive groups may be chosen from those described above and may be coupled directly or via a linker. In a particular embodiment, an oligonucleotide molecule containing a reactive amino group is reacted with iodoacetamide activated with 1,1′-carbonyldiimidazole and then conjugated to the pyridine reactive group of the polymer poly(4-vinylpyridine).

In some embodiments, a tag (oligonucleotide or non-oligonucleotide) which is not associated with the polymer is removed by purification. Exemplary purification methods include, but are not limited to, precipitation, filtration through an anion exchange membrane, ultrafiltration, diafiltration, and gel permeation chromatography.

In some embodiments, the biomolecule of interest is selected from the group consisting of a protein (e.g., a recombinant protein), an antibody and a vaccine. In some embodiments, the antibody is a monoclonal antibody.

In a particular embodiment, a method according to the present invention uses an oligonucleotide molecule comprising the sequence set forth in SEQ ID NO:1.

In some embodiments, the oligonucleotide molecule comprises a nucleotide sequence predicted to be free of secondary structure.

In some embodiments, the oligonucleotide molecule comprises a nucleotide sequence free of homopolymers of four or more bases in length.

In some embodiments, the polymer is poly(4-vinyl pyridine). In other embodiments, the polymer is polyvinylamine. In still other embodiments, the polymer is polyallylamine. In still further embodiments, the polymer is a polyallylamine or polyvinylamine polymer modified with a hydrophobic group. In a particular embodiment, the polyallylamine has a molecular weight of 150 kDa, where 30% of its amine groups are covalently modified through a reaction with benzylchloride.

In some embodiments the polymer is responsive to addition of multivalent anions, for example, phosphate or citrate ions.

In some embodiments, a tag used for detection is a fluorescent tag or a fluorophore, which may be attached to a polymer or to an oligonucleotide molecule which binds the polymer. Alternatively, it may be attached to a probe which hybridizes to the oligonucleotide molecule.

Exemplary fluorescent tags include, but are not limited to, N-(4,4-difluoro-1,3,5,7-tetramethyl-4-bora-3a,4a-diaza-s-indacene-2-yl)iodoacetamide (also known as BODIPY® 507/545 IA (INVITROGEN)) and 7-hydroxycoumarin-3-carboxylic acid, succinimidyl ester (INVITROGEN).

In some embodiments, the polymer bearing a covalently attached fluorescent molecule or fluorophore is detected and quantified via fluorescence spectrophotometry.

In some embodiments, a tag used for detection is a radioactive molecule, which may be attached to a polymer or to an oligonucleotide molecule which hinds the polymer. Alternatively, it may be attached to a probe which hybridizes to the oligonucleotide molecule.

Exemplary of radioactive tags include, but are not limited to, methyl iodide [3H] and methyl iodide [14C] (MP BIOMEDICALS).

In some embodiments, the polymer bearing a covalently attached radioactive group is detected and quantified using a scintillation counter.

In some embodiments, a tag used for detection is a hapten molecule which may be attached to a polymer or to an oligonucleotide molecule which binds the polymer. Alternatively, it may be attached to a probe which hybridizes to the oligonucleotide molecule. Exemplary hapten tags include, but are not limited to, fluorescein, biotin, and dinitrophenol. Examples hapten tags including a reactive group include DNP—X, SE, 6-(2,4-dinitrophenyl)aminohexanoic acid, succinimidyl ester (INVITROGEN), USB-X™ biotin C₂-iodoacetamide (desthiobiotin-X C2-iodoacetamide) (INVITROGEN), and 5(6)-fluorescein isothiocyanate mixed isomer (THERMO SCIENTIFIC).

In some embodiments, a polymer containing an attached hapten is detected and quantified using an immunoassay, such as an ELISA. An exemplary example of an ELISA is the Biotin ELISA Kit (ALPCO DIAGNOSTICS).

In some embodiments, a polymer containing a covalently attached hapten is detected and quantified using an avidin/streptavidin detection method known in the art or use of enzyme reporters and amplification techniques (e.g., horseradish peroxidase, alkaline phosphatase), tyramide signal amplification or fluorescent probes.

In some embodiments according to the methods of the invention, the one or more impurities are selected from the group consisting of whole cells, cell fragments, lipids, DNA, RNA, host cell proteins, endotoxins and viruses.

### BRIEF DESCRIPTION OF THE DRAWINGS

FIG. 1 depicts a schematic of a purification process as applicable to a pH dependent polymer such as poly(4-vinylpyridine), which has binding affinity for a desired biomolecule of interest.

FIG. 2 depicts an industry standard template for the purification of monoclonal antibodies.

FIG. 3 is a graph depicting a quantitative PCR calibration curve for un-conjugated oligonucleotide tag. The circled point is for ˜10 copies of tag input as determined from dilution of a stock of an oligonucleotide of unknown weight resuspended in a known volume.

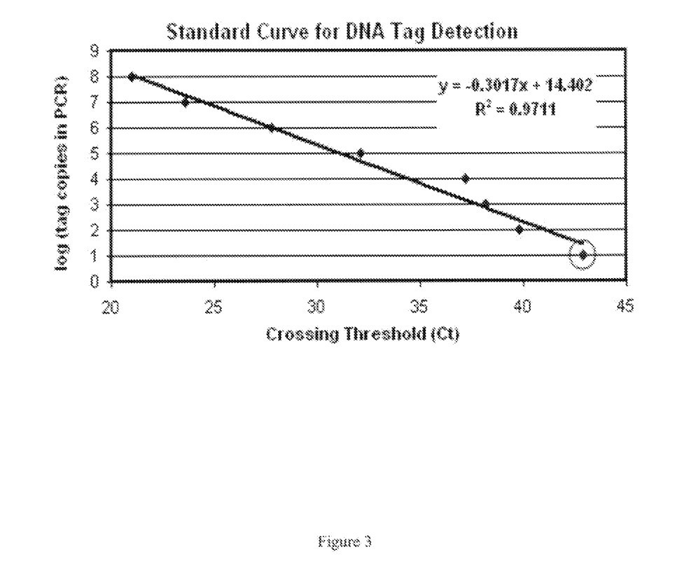

FIG. 4 is a graph depicting the characterization of the molar ratio of an oligonucleotide tag to polymer in the oligonucleotide-conjugated polymer. Open diamonds show readout obtained with un-conjugated oligonucleotide tag, closed diamonds show readout obtained with standard workup of the oligonucleotide tag-conjugated polymer and the closed triangles show the readout obtained with the oligonucleotide tag-conjugated polymer purified by precipitation from reaction mixture in dimethyl formamide using ethyl acetate, followed by resuspension in methanol and re-precipitated with 0.1 M sodium hydroxide. Based on the assumption that the sensitivity for conjugated and un-conjugated oligonucleotide tag is similar, the sensitivity for conjugated oligonucleotide-tagged polymer is ˜125 parts per trillion or lower for the experimentally determined conjugation ratio of 1 molecule of tag per 500 molecules of polymer.

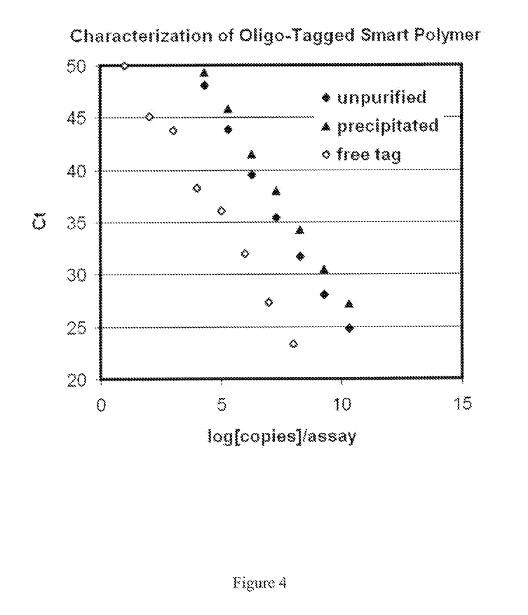

FIG. 5 is a schematic depicting the use of oligonucleotide tag-conjugated polymers for downstream process monitoring. The process workflow is shown where the oligonucleotide-tag conjugated polymer is used for simultaneous clarification and purification where the polymer binds both the biomolecule of interest as well as one or more impurities, where the biomolecule of interest is subsequently selectively eluted, whereas the one or more impurities remain bound to the polymer.

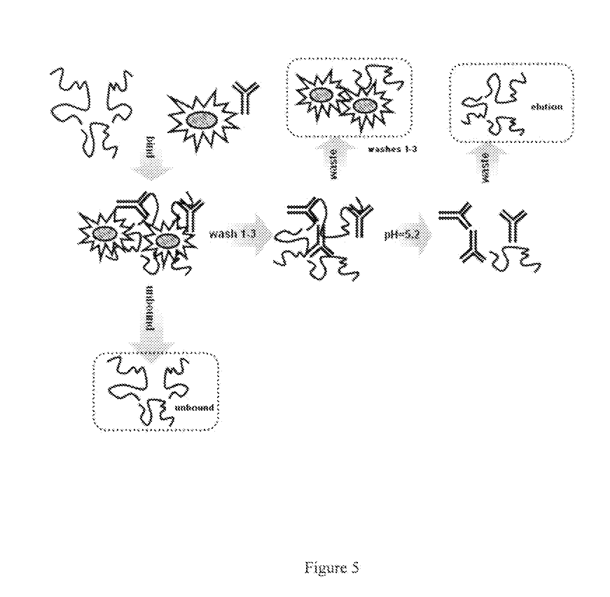

FIG. 6 is a graph depicting the analysis of various fractions from the workflow in FIG. 5 for residual polymer using readout from the oligonucleotide tag tracer. As the fractions move through the purification process, the curves begin to flatten out with a crossing threshold (Ct) of around 48 for all dilutions, similar to the no tag control.

FIG. 7 depicts a standard curve for quantifying unknown concentrations of a BODIPY 507/545 tagged polymer, which was generated using known amounts of a BODIPY 507/545 tagged, hydrophobically modified polyallylamine stimulus responsive polymer. The concentration of polymer in parts per million (ppm) is shown on the x-axis and intensity at 545 nm is shown on the y-axis.

FIG. 8 is a graph depicting the quantification of a BODIPY 507/545 tagged, hydrophobically modified polyallylamine stimulus responsive polymer remaining after flocculation and clarification of a cell culture medium. The flocculant dose % (weight of polymer/volume of cell culture fluid) that was added is shown on the x-axis and the residual polymer detected in the filtrate is shown on the y-axis in parts per million.

### DETAILED DESCRIPTION OF THE INVENTION

The present invention provides methods of detecting residual amounts of a polymer in a sample, where the polymer is used for separating a biomolecule of interest from one or more impurities. In some embodiments, such a polymer is “a stimulus responsive polymer” or “a smart polymer.” In some embodiments, the smart polymer binds a biomolecule of interest. In other embodiments, the smart polymer binds one or more impurities. In still other embodiments, the smart polymer binds both a biomolecule of interest and one or more impurities, where the biomolecule of interest is subsequently eluted from the polymer and the one or more impurities remain bound to the polymer.

In order that the present disclosure may be more readily understood, certain terms are first defined. Additional definitions are set forth throughout the detailed description.

### I. Definitions

As used herein, the term “biomolecule of interest” or “desired biomolecule of interest,” refers to a biological material (e.g., a target biomolecule or a product of interest) which is being separated from one or more impurities using a polymer, e.g., a stimulus responsive polymer. Exemplary biomolecules of interest include, for example, proteins (e.g., recombinant proteins), antibodies and vaccines. In a particular embodiment, a biomolecule of interest is a monoclonal antibody. In some embodiments, methods according to the present invention detect residual amounts of a polymer which may be remaining after binding and precipitating (not necessarily in that order) a biomolecule of interest, thereby to separate it from one or more impurities. In other embodiments, the methods according to the present invention detect residual amounts of a polymer which may be remaining in a sample after binding and precipitating (not necessarily in that order) one or more impurities, thereby to separate a biomolecule of interest from the one or more impurities.

As used herein, the term “one or more impurities,” as used herein, refers to any foreign or objectionable molecule, including a whole cell, cell fragment or a biological macromolecule such as a DNA, an RNA, one or more host cell proteins, endotoxins, lipids and one or more additives which may be present in a sample containing a biomolecule of interest that is being separated from such a foreign or objectionable molecule. In some embodiments according to the present invention, one or more impurities are removed from a sample containing a biomolecule of interest and one or more impurities using a polymer. Methods according to the invention enable detection of residual amounts of a polymer which may be used for the separation of one or more impurities from a biomolecule of interest.

The term “polymer” as used herein, refers to a molecule formed by covalent linkage of two or more monomer units. These monomer units can be synthetic or occur in nature. The polymers formed by the repeating units can be linear or branched. Examples of polymers include, but are not limited to, polyethylene glycol, polypropylene glycol, polyethylene, polyallylamine, polyvinylalcohol, polystyrene and copolymers (e.g. polystyrene-co-polypyridine, polyacrylic acid-co-methyl methacrylate, pluronics, PF68 etc). In some embodiments according to the present invention, polymers comprise a polytelectrolyte backbone. Also described herein are copolymers, which may be used in the methods according to the present invention, where the copolymers are responsive to a stimulus. In general, it is understood that in case of polymers, the monomeric units are of the same type, whereas, a copolymer will usually have different types of monomeric units.

The term “stimulus responsive polymer,” as used herein, is a polymer or copolymer which exhibits a change in a physical and/or chemical property after the addition of a stimulus. A typical stimulus response is a change in the polymer's solubility.

In some embodiments, a stimulus responsive polymer is soluble under a certain set of process conditions such as pH, salt concentration or temperature and is rendered insoluble and precipitates out of solution upon a change in conditions (temperature, salt concentration or pH), e.g., as a response to the change in one or more conditions. For example, the polymer poly(N-isopropylacrylamide) is water soluble at temperatures below about 35° C., but become insoluble in water at temperatures of about 35° C. Smart polymers may be used for binding one or more impurities or they may be used for binding a biomolecule of interest in a sample containing the biomolecule of interest and one or more impurities. Accordingly, in some embodiments, methods according to the invention may be used for the detection of residual amounts of a smart polymer which may remain after the binding and/or precipitation of the biomolecule of interest with the smart polymer. In some embodiments, residual amounts of smart polymer are detected after elution of the biomolecule of interest from the polymer. In other embodiments, residual amounts of a smart polymer are detected after binding and/or precipitation of one or more impurities with the smart polymer.

The term “stimulus” or “stimuli,” as used interchangeably herein, is meant to refer to a physical or chemical change in the environment which results in a response by a stimulus responsive polymer according to the present invention. Accordingly, the present invention provides methods of detecting residual amounts of polymers which are responsive to a stimulus which results in a change in the solubility of the polymer. Examples of stimuli to which polymers may be responsive, include, but are not limited to, e.g., changes in temperature, changes in conductivity, changes in electric and/or magnetic field and/or changes in pH. In some embodiments, a stimulus comprises addition of a complexing agent or a complex forming salt to a sample. In various embodiments, a stimulus is generally added after the addition of a polymer to a sample. Although, the stimulus may also be added during or before addition of a polymer to a sample.

In a particular embodiment, a stimulus is a salt. Exemplary salts include, but are not limited to, multivalent ions such as, e.g., citrate, phosphate, sulfate and EDTA, and ion-association salts such as perchlorate, dodecyl sulfate sodium salt, dodecyl benzene sulfate, Fe(II)-4-chloro-2-nitrophenol anion, tetraphenyl borate sodium salt and hexanitrodiphenol amine (see. e.g., ANALYTICAL SCIENCES, DECEMBER 1987, VOL. 3, p. 479). In general, one of ordinary skill in the art would be familiar with numerous salts which are known in the art and may be used as a stimulus with the polymers described herein.

In various embodiments described herein, a stimulus such as, e.g., a salt, is added to a sample containing a biomolecule of interest and one or more impurities following the addition of a polymer which binds one or both of the biomolecule of interest and/or one or more impurities. The addition of a stimulus, e.g., a salt, results in the precipitation of free polymer as well as both soluble and insoluble complexes of the polymer and one or both of the biomolecule of interest and/or one and more impurities.

The amount of a salt which is required to induce precipitation of the soluble or insoluble complexes depends on factors such as, e.g., polymer concentration and concentration of biomolecule of interest or one or more impurities which the polymer binds. For example, some polyelectrolytes such as polyallylamine have a charge density which varies with pH (level of amine protonation). As the pH is increased, the level of charge density is reduced so the degree of stimulus required to induce precipitation will be different than the degree of stimulus at a lower pH or higher charge density state.

As used herein, the terms “enriching,” “separating,” “isolating” and “purifying” refers to the method of using a polymer to purify one or more desired biomolecules from a sample or a biological material containing stream. In various embodiments, enriching, separating, isolating or purifying a biomolecule of interest encompasses removal of one or more impurities present in a sample containing the biomolecule of interest. A biomolecule of interest may be separated from one or more impurities either by using a polymer which binds the one or more impurities or by using a polymer which binds the biomolecule of interest. In some embodiments, a polymer binds both a biomolecule of interest as well as one or more impurities, where the biomolecule of interest is subsequently selectively eluted from the polymer, whereas the one or more impurities remain bound.

As used herein, the term “residual amount” or “residual amounts” refers to an amount of polymer used for purification of biological materials, e.g., a stimulus responsive polymer or a polymer used for flocculation, which amount is equal to or less than the amount which will remain dissolved and/or dispersed in a sample or a biological material containing stream after the polymer binds and precipitates one or more impurities or binds and precipitates the desired biomolecule of interest. The residual amount also refers to an amount of polymer, e.g., a stimulus responsive polymer, which amount is equal to or less than the amount which will remain dissolved and/or dispersed in a sample or a biological material containing stream after a stimulus is applied, such that the polymer hinds and precipitates or precipitates and binds one or more impurities or a desired biomolecule of interest.

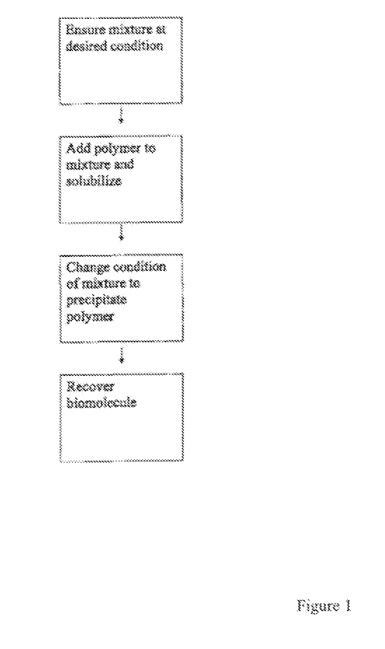

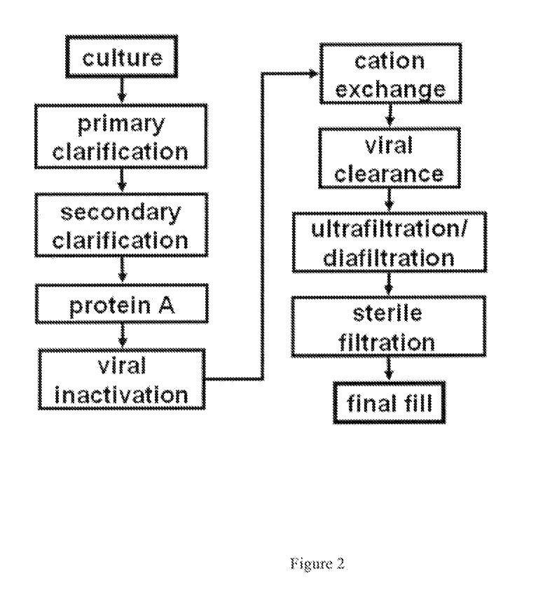

As used herein, the term “composition”, “solution” or “sample” refers to a biological material containing stream. Typically the biological material containing stream refers to a mixture of a biomolecule of interest or a desired product to be purified along with one or more undesirable entities or impurities. In some embodiments, the sample comprises a biomolecule of interest (e.g., a therapeutic protein or an antibody) along with one or more impurities (e.g., host cell proteins, DNA, RNA, lipids, cell culture additives, cells and cellular debris). Generally, the biological material containing stream may be subjected to a purification scheme, as depicted in FIG. 1. In some embodiments, the sample comprises feedstock or cell culture media into which a biomolecule of interest or a desired product is secreted. In some embodiments, a sample from which a biomolecule is to be purified using one or more stimulus responsive polymers described herein is “partially purified” prior to contacting the sample with a stimulus responsive polymer. Partial purification may be accomplished, for example, by subjecting the sample to one or more purification steps, such as, e.g., one or more non-affinity chromatography steps such as, for example, depicted in FIG. 2. The biomolecule may be separated from one or more undesirable entities or impurities either by precipitating the one or more impurities or by precipitating the biomolecule of interest.

In sonic embodiments, a stimulus responsive polymer selectively and reversibly binds to a biomolecule of interest under a first set of conditions and precipitates the biomolecule of interest under a second set of conditions, e.g., upon the addition of a stimulus to the sample. In other embodiments, a stimulus responsive polymer selectively binds to one or more impurities under a first set of conditions and precipitates the one or more impurities under a second set of conditions. In sonic embodiments, a stimulus responsive polymer selectively binds and precipitates one or more of host cell proteins, DNA, whole cells, cellular debris, viruses and/or cell culture additives, upon the addition of a stimulus. In still other embodiments, a stimulus responsive polymer binds both a biomolecule of interest as well as one or more impurities under a first set of conditions and where the biomolecule of interest is selectively eluted from the polymer under a second set of conditions, whereas the one or more impurities remain bound.

The term “precipitate,” precipitating” or “precipitation,” as used herein, refers to the alteration of a bound (e.g., in a complex with a biomolecule of interest or one or more impurities) or unbound polymer from an aqueous and/or soluble state to a non-aqueous and/or insoluble state.

The term “tag” as used herein, refers to any molecule which is capable of being bound to a polymer, as used herein, and can be detected using suitable means. In one embodiment, a tag used in the detection methods according to the claimed invention is an oligonucleotide molecule, which can be detected using amplification means or non-amplification means. In another embodiment, a tag used in the detection methods according to the claimed invention is a hapten molecule, a fluorescent molecule or a radioactive molecule, which is directly attached to the polymer and can be readily detected using suitable means well known in the art and those described herein. Further, in certain embodiments, instead of a hapten molecule (e.g., biotin), a radioactive molecule or a fluorescent molecule being attached to a polymer, they are attached to the oligonucleotide molecule, which in turn is attached to the polymer. In further embodiments, a hapten molecule, a fluorescent molecule or a radioactive molecule are attached to a probe which hybridizes to an oligonucleotide molecule attached to a polymer, and can be subsequently detected.

As used herein, the term “oligonucleotide tag” or “oligonucleotide molecule,” refers to a nucleic acid molecule which is capable of binding to a polymer and is used for the detection of that polymer. In a particular embodiment, the oligonucleotide tag used in the methods according to the present invention comprises the sequence set forth in SEQ ID NO:1. It is desirable that the sequence of the oligonucleotide tag or molecule used in the methods of the present invention does not otherwise appear in the various components of the biological material containing stream. One of ordinary skill in the art will be readily able to verify the presence or the absence of the sequence of the oligonucleotide tag, e.g., using computational (e.g., BLAST against host cell DNA sequence) or experimental methods (e.g., hybridization techniques) well known in the art. In some embodiments, the association between the polymer and the oligonucleotide molecule comprises covalent attachment. In some embodiments, the oligonucleotide molecule is detected using an amplification reaction. In other embodiments, the oligonucleotide molecule is detected using a non-amplification reaction, e.g., in instances where the oligonucleotide molecule is itself labeled with a detectable moiety such as, for example, a hapten molecule (e.g., biotin), a fluorescent molecule or a radioactive molecule. Further, hybridization methods using, e.g., a probe, may be used for the detection of the oligonucleotide molecule. In some embodiments, a probe is labeled with a hapten molecule, a fluorescent molecule or a radioactive molecule.

As used herein, the term “primer” refers to a single-stranded nucleic acid molecule that is capable of hybridizing to an oligonucleotide molecule or its reverse complement and can participate in an amplification reaction (e.g., polymerase chain reaction) under appropriate conditions. In some embodiments, an oligonucleotide molecule is detected using a set of 5′ and 3′ primes which are capable of hybridizing to an oligonucleotide molecule and can be used for the detection of the oligonucleotide molecule. The amount of the oligonucleotide molecule detected is indicative of the amount of residual polymer present in a sample.

As used herein, the term “probe” refers to a single-stranded nucleic acid molecule which is capable of hybridizing to an oligonucleotide molecule e.g., under stringent hybridization conditions. In some embodiments, the probe is a labeled probe, in that it has a detectable signal attached to it. In some embodiments, the detectable signal is a label, e.g., a dye. In some embodiments, the amount of the probe detected is indicative of the amount of residual polymer present in a sample. In some embodiments, the probe includes a hapten molecule, a radioactive molecule or a fluorescent molecule, which can be detected.

As used herein, the term “capable of hybridizing” means that Watson-Crick-type hydrogen bonds can be formed between anti-parallel nucleic acid strands that favor a duplex state for the nucleic acid strands under given conditions, e.g., stringent hybridization conditions.

As used herein, the term “detecting the tag” refers to a method that allows measurement and quantitation of a tag, as used herein. In some embodiments, where the tag is an oligonucleotide molecule, detection comprises use of an amplification reaction (e.g., using a polymerase chain reaction). In other embodiments when the tag is an oligonucleotide molecule, detection comprises use of a non-amplification reaction (e.g., using a labeled probe or use of a detectable moiety such as, a hapten molecule, a fluorescent molecule or a radioactive molecule attached to the oligonucleotide molecule or to the probe). In still other embodiments, a tag is attached to a polymer instead of an oligonucleotide, which can subsequently be detected. Accordingly, a detectable moiety including, but not limited to, e.g., a hapten molecule, a fluorescent molecule or a radioactive molecule may be attached to a polymer.

As used herein, the term “amplification reaction” refers to a methodology whereby an initial number of molecules are increased to a greater number of molecules. In some embodiments, amplification reaction employs polymerase chain reaction.

As used herein, the term “reactive group” refers to a set of atoms in a molecule that react preferentially with another molecule over atoms in the remainder of the molecule under a given set of conditions.

As used herein, the term “secondary structure” of an oligonucleotide molecule, as used herein, refers to a three-dimensional form created by basepairing interactions within a single nucleic acid molecule. Generally, in case of nucleic acid molecules, the secondary structure is created by the hydrogen bonding between the nitrogenous bases. In various embodiments according to the present invention, it is desirable that an oligonucleotide molecule used in the methods according to the present invention is free of secondary structure. Secondary structure in a nucleic acid molecule can be easily predicted using one or more software tools well known to one of ordinary skill in the art and those described herein.

### II. Exemplary Processes for Purifying a Biomolecule of Interest Utilizing a Polymer

In general, a biomolecule of interest such as, for example, a protein (e.g., a recombinant protein or an antibody), may be purified using a method which employs expression of the protein in a host cell (e.g., by using a host cell which includes a nucleic acid molecule which encodes the protein) followed by the purification of the protein.

In some instances, a desired host cell line is grown in a bioreactor in order to express the protein at desirable levels. Exemplary host cell lines include Chinese hamster ovary (CHO) cells, myeloma (NSO) cells, bacterial cells such as E. coli and insect cells. Once a protein is expressed at the desired levels, the protein is removed from the host cell and harvested. Suspended particulates, such as cells, cell fragments, lipids and other insoluble matter are typically removed from the protein-containing fluid by filtration or centrifugation, resulting in a clarified fluid containing the protein of interest in solution as well as other soluble impurities.

The second step typically involves the purification of the harvested protein to remove one or more impurities which are inherent to the process. Examples of such impurities include, e.g., host cell proteins (HCP, which are proteins other than the desired or targeted protein), nucleic acids, endotoxins, viruses, and protein aggregates. This purification process typically involves several chromatography steps, which can include affinity, ion exchange hydrophobic interaction, etc. on solid matrices such as porous agarose, polymeric or glass. An exemplary purification process is depicted in FIG. 2.

Other alternative methods for purifying proteins have been investigated in recent years. One such method involves a flocculation technique (see, e.g., WO2008/091740). In this technique, a soluble polyelectrolyte is added to a clarified or unclarified cell culture broth to capture the impurities thereby to form a flocculant, which is allowed to settle and can be subsequently removed from the protein solution.

In co-pending U.S. Patent Publication No. 20090036651, incorporated by reference herein in its entirety, a polymer, soluble under certain conditions, such as temperature or pH, is used to selectively bind one or more impurities while in its soluble state and is then precipitated out upon a change in condition (pH or temperature, etc) removing the one or more impurities with it. The biomolecule of interest can be further subjected to traditional chromatography or membrane absorbers and the like.

FIG. 1 depicts a schematic of a non-limiting purification process as applied to a pH dependent polymer such as poly(4-vinylpyridine), which is capable of binding one or more impurities. In the first step, the mixture is conditioned to the correct parameter(s) to maintain the polymer of choice in solution. Alternatively, if the conditions of the mixture are already such that the polymer(s) become soluble in the mixture, no further conditioning may be required. Also, the polymer(s) may be added as a solid to an unconditioned mixture and then the mixture (containing the solid polymer(s)) may be conditioned to the correct parameters to dissolve the polymer(s) in the mixture. In the second step, the polymer(s) is added to the mixture and caused to go into solution in order to contact the various constituents of the mixture. It is contemplated that the second step may occur in a bioreactor, e.g., if the bioreactor is a disposable item or in a separate holding tank, as desired. In a third step, the mixture conditions are changed to cause the polymer(s) to precipitate out of solution while retaining one or more entities of the mixture with it. The mixture and the precipitated polymer(s) are then separated from each other in the fourth step. The precipitate and remaining mixture may be separated by centrifugation or filtration.

In some embodiments, once a cell culture fluid is clarified, the biomolecule of interest may be subjected to one or more known additional process steps such as chromatography steps including, but not limited to, ion exchange, hydrophobic interaction or affinity performance tangential flow filtration (HPTFF) with or without charged UF membranes, viral removal/inactivation steps, final filtration steps and the like.

One undesired outcome of using polymers in the purification of a biomolecule of interest from a biological containing stream is the possibility that an undefined amount of polymer may remain in the stream. If desired, one may conduct one or more additional steps to ensure that all polymer has been removed from the mixture by subjecting the mixture to a step containing a material that will remove any residual polymer from the mixture such as ion exchange resin, activated carbon, alumina, diatomaceous earth and the like. However, it is desirable to have a sensitive assay to quantify residual polymer amounts and track the efficiency of the steps described above in removing any residual polymer. The methods according to the present invention provide improved and sensitive assays to detect and quantify residual amounts of polymers.

Without wishing to be bound by theory, it is understood that the detection methods according to the claimed invention may be used in conjunction with any purification process which employs a polymer for precipitating one or both of a biomolecule of interest and/or one or more impurities, where a detectable oligonucleotide or non-oligonucleotide tag, as descried herein, can be attached to the polymer.

### III. Exemplary Polymers for Use in Methods of Purifying a Biomolecule of Interest

Polymers including those described in U.S. Patent Publication Nos. 20080255027, 20090036651, 20090232737 and 20110020327, as well as U.S. patent application Ser. No. 13/108,576, each of which are incorporated by reference in their entirety herein, may be used in methods described herein.

Exemplary polymers include, but are not limited to, poly(N-isopropylacrylamide), agarose, dextran, polyethylene oxide, hydroxyalkyl cellulose, cationic polyelectrolytes chosen from the group containing chitosan, polyvinylpyridine, copolymers of vinylpyridine, primary amine containing polymers, secondary amine containing polymers, tertiary amine containing polymers, and anionic polyelectrolytes chosen from the group containing copolymers of acrylic acid and methyl methacrylate and copolymers of methacrylic acid and methyl methacrylate.

One or more polymers such as, e.g., poly(N-vinyl caprolactam), poly(N-acryloylpiperidine), poly(N-vinylisobutyramide), poly(N-substituted acrylamide) (e.g., poly(N-isopropylacrylamide), poly(N,N-diethylacrylamide), and poly(N-acryloyl-N-alkylpiperazine), hydroxyalkylcellulose, copolymers of acrylic acid and methacrylic acid, polymers and copolymers of 2 or 4-vinylpyridine and chitosan may be used in the methods described herein, either with or without a ligand or a functional group attached to the polymer, in order to selectively and reversibly bind to a biomolecule of interest or one or more impurities, thereby to separate the biomolecule of interest from one or more impurities in a sample, e.g., a biological material containing stream.

In a particular embodiment, a polymer used in the methods according to the claimed invention is a polyvinylamine polymer. In another embodiment, a polymer used in the methods according to the claimed invention is a polyallylamine polymer. In yet another embodiment, a polymer used in the methods according to the claimed invention is a polyallylamine polymer modified with a hydrophobic group, e.g., as described in U.S. patent application Ser. No. 13/108,576, filed on May 16, 2011, the entire content of which is incorporated by reference herein in its entirety.

### IV. Methods of Identifying Oligonucleotide Molecules for Attaching to a Polymer

In some methods of detecting residual polymer described herein, an oligonucleotide molecule may be employed. A library of potential oligonucleotide molecule sequences 40 nucleotides in length, having a nearly equal nucleotide base distribution A:C:G:T˜1:1:1:1) and devoid of 4 or more contiguous C or G bases may be generated in silico, e.g., using a software program. Once a potential oligonucleotide sequence is identified, it is desirable that the sequence does not appear in the biological material containing stream. Accordingly, the presence or the absence of the sequence can be verified using computational (e.g., by running BLAST searches against the genomic sequence of host cell DNA) and/or experimental (e.g., by hybridization against the biological material containing stream) techniques.

### V. Methods of Tagging a Polymer with an Oligonucleotide Molecule

Oligonucleotides may be synthesized with internal or terminal reactive groups such as halogen, epoxy, hydroxyl, amino, carboxylic acid or sulfhydryl, which are subsequently attached to a polymer. Further, oligonucleotides with internal or terminal reactive groups may be further activated with various coupling chemistries such as, e.g., cyanogen bromide, N-hydroxy Succinimide esters, 1,1′-carbonyl diimidazole, carbodiimide EDC, glycidyl, organic sulfonyl chlorides, azlactones, cyanuric chloride, and maleimide, in order to facilitate attachment to a polymer. See, e.g. Immobilized affinity ligand techniques. Hermanson, Mania and Smith 1992.

In some embodiments, for oligonucleotides containing a 3′-terminal amino group, iodoacetamide activated with 1,1′-carbonyldiimidazole may be used to attach a terminal iodine group that subsequently reacts with pyridine in polyvinylpyridine resulting in covalent attachment of the oligonucleotide to the polymer. Epichlorohydrin may also be used to attach a terminal glycidyl group to oligonucleotides containing a 3′-terminal amino group that subsequently reacts with pyridine in polyvinylpyridine resulting in covalent attachment of the oligonucleotide to the polymer. The specific embodiments described herein are not meant to be limiting in any way.

### VI. Methods of Tagging a Polymer with a Non-Oligonucleotide Molecule

In some embodiments, polymers are tagged with a non-oligonucleotide molecule such as, e.g., a radioactive molecule, a fluorescent molecule or a hapten molecule. In some embodiments, the molecules have a terminal or internal reactive groups that can react directly with the polymer resulting in covalent conjugation. In one embodiment, the terminal or internal group of the molecule maybe activated by known protein chemistry methods, e.g., formation of N-hydroxysuccinimidyl ester (NHS-ester), acid fluoride, tetrafluorophenyl ester (TFP-ester), or STP-ester, such that it reacts with the amino or pyridine function of the polymer with the formation of an acid amide (see, e.g., Immobilized affinity ligand techniques. Hermanson, Mallia and Smith 1992). The coupling reaction may be performed in organic solutions such as methanol, dimethyl formamide (DMF) or dimethyl sulfoxide (DMSO) or aqueous solutions at pH between pH 5 and pH 12. In one embodiment, the coupling reaction may be performed at room temperature (about 20° C. to about 60° C.).

Examples of fluorescent molecules which may be used as tags in the methods described herein include, but are not limited to, 4-acetamido-4′-isothiocyanatostilbene-2,2′ disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; alexa; fluorescien; conjugated multi-dyes; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Atto dyes, Cy3; Cy5; Cy5.5; Cy7; IRD 700; LRD 800; La Jolla Blue; phthalo cyanine; and naphthalo cyanine. Additionally, optically-detectable moieties may also be used as tags in the methods of the invention.

Examples of radioactive molecules that may be used as tags are isotope containing molecules, including but are not limited to, ¹⁴C, ³H, ⁴⁵Ca, ⁵¹Cr, ⁵⁷Co, ³⁶Cl, ¹²⁵I, ³²P, ³³P, and ³⁵S.

Examples of hapten molecules that may be used as tags include DNP—X, SE, 6-(2,4-dinitrophenyl)aminohexanoic acid, succinimidyl ester (INVITROGEN), DSB-XT™ biotin C₂-iodoacetamide (desthiobiotin-X C2-iodoacetamide) (INVITROGEN), and 5(6)-fluorescein isothiocyanate mixed isomer (THERMO SCIENTIFIC).

### VII. Methods of Removing Unassociated Tag

Following the tagging of a polymer with a tag, it is desirable to remove any unassociated tag from the sample. For example, the DNA tagged PVP solution can be purified for removal of un-conjugated DNA tags by filtration through an anion exchange membrane, e.g., Chromasorb® (MILLIPORE, Billerica, Mass.), following pre-equilibration with a solution or DMF and water (50:50 volume ratio).

Removal of un-conjugated tags (oligonucleotide or non-oligonucleotide) from a solution can be achieved via precipitation. For example, a solvent is mixed with the tagged polymer containing solution at a specific ratio so as to retain the unconjugated tag in the supernatant while the tagged polymer precipitates. Alternatively, suitable conditions can be selected which facilitate retention of the tagged polymer while precipitating the unconjugated tag. The skilled artisan will be able to readily identify suitable conditions for precipitation based on knowledge in the art coupled with routine experimentation.

Ultrafiltration/diafiltration membranes may be selected based on nominal molecular weight cut-off (“NMWCO”) so as to retain the product of interest in the retentate, while allowing low molecular weight materials such as un-associated tags to pass into the filtrate. One skilled in the art will be able to select such membranes based on the size and nature of the product of interest, coupled with routine experimentation. In a particular embodiment, where the tag is an oligonucleotide 10-60 bases in size, and the polymer is poly(4-vinyl pyridine) 200,000 Da in size, ultrafiltration/diafiltration can be performed using a Millipore Centricon Plus-70® unit having a NMWCO of 30. Gel filtration chromatography columns can be selected based on the exclusion molecular weight so as large molecular weight materials such as, for example polymers, spend less time in the pore structure of the stationary phase and are eluted faster than the smaller molecular weight materials, such as, for example, un-associated oligonucleotides, which spend more time in the pore structure and elute at a later time. One skilled in the art will be able to readily select such columns based on the size and nature of the material of interest, coupled with no more than routine experimentation.

In a particular embodiment, where the tag is an oligonucleotide 10-60 bases in size, and the polymer is poly(4-vinyl pyridine) 200,000 kDa in size, gel permeation chromatography can be performed using a SHOWA DENKO K.K GPC KF-804L column having an exclusion molecular weight of 400,000 Da.

### VIII. Methods of Detecting an Oligonucleotide Molecule Associated with a Polymer

Following the attachment of an oligonucleotide molecule onto a polymer and the subsequent removal of any unassociated molecule, as described herein, the associated molecule can be detected using an amplification reaction or a non-amplification reaction. In case of an amplification reaction, primers are selected which hybridize to the 5′ and 3′ portions of the molecule. For example, primers may be selected that allow the molecule sequence, in free or polymer-conjugated form, to be amplified and detected using an amplification reaction, e.g., polymerase chain reaction (PCR). In some embodiments, amplification products may be visualized by direct measurement of fluorescence based on the molecule to obtain a crossing threshold value (O). The measured Ct value may be used to determine the concentration of molecule present in sample using a calibration curve created from known concentrations of molecule.

In case of a non-amplification reaction, a labeled probe may be used for the detection of the oligonucleotide molecule. Alternatively, the oligonucleotide molecule may itself be labeled with a detectable moiety such as, for example, a hapten tag, a fluorescent tag or a radioactive tag. In some embodiments, a probe which hybridizes with the oligonucleotide molecule is labeled with a detectable moiety.

### VIII. Methods of Detecting a Non-Oligonucleotide Molecule Associated with a Polymer

Following the attachment of a non-oligonucleotide molecule onto a polymer such as, for example, a fluorescent tag, a radioactive tag or a hapten tag, and the subsequent removal of any unassociated molecule: the associated molecule can be detected by using any of a variety of techniques depending upon the molecule which is used as a tag. Exemplary detection methods include radioactive detection, optical absorbance detection, e.g., UV-visible absorbance detection, optical emission detection, e.g., fluorescence or chemiluminescence, immunoassay, e.g., ELISA, and avidin/streptavidin detection methods. Devices capable of sensing fluorescence from a single molecule include scanning tunneling microscope (siM) and the atomic force microscope (AFM). For radioactive signals, a phosphorimager device can be used. Other commercial suppliers of imaging instruments include General Scanning Inc. (Watertown, Mass.), Genix Technologies (Waterloo, Ontario, Canada) and Applied Precision Inc.

This invention is further illustrated by the following examples which should not be construed as limiting. The contents of all references, patents and published patent applications cited throughout this application, as well as the Figures, are incorporated herein by reference.

### EXAMPLES

### Example I

### Selection of Oligonucleotide Sequence for Conjugation to Polymer

A library of 50 or more potential oligonucleotide tag sequences of 40 bases in length, with nearly equal nucleotide base distribution (A:C:G:T˜1:1:1:1) and devoid of 4 or more contiguous C or G bases were generated in silico, e.g., using software.

Members of the oligonucleotide tag library were screened for secondary structure using the program M-fold (Zuker M. 2003. Mfold web server for nucleic acid folding and hybridization prediction. Nucleic Acids Res. 31: 3406) choosing prediction for linear DNA at 37° C., 1 mM MgCl2, and 50 mM NaCl and prioritized by the melting point of predicted structures, lower being better, as shown in Table I below.

Selected oligonucleotide tag sequences are subsequently chemically synthesized with a variety of internal or terminal reactive groups for conjugation to polymer.

TABLE 1

| ID | Sequence | % GC | hybrid Tm | hairpin Tm |
|---|---|---|---|---|
| 38 | CCAATTAGAGAGGGCCGGATTAAAGGTAAGCTATTCCATG | 45 | 76.9° C. | 31.4° C. |
| 46 | AATGAGTAGATTCAATCCGAGGGTTAGGTATCTAGTTGAC | 40 | 74.8° C. | 33.8° C. |
| 36 | GTTATCGGATTCTTAATAGGAACACACTCTATCTCACCCG | 42 | 75.8° C. | 34.0° C. |
| 44 | TATTTCTCCATAATACTTGGCCGAGAGTAATTAAGCCGGG | 42 | 75.8° C. | 34.0° C. |
| 5 | CTTTAGCAAGTACATGGGTCGGAGGTGGGAGAATGTTGTA | 47 | 77.9° C. | 35.5° C. |
| 20 | TAACAGATCCAGCACGCCATCTTCTATAGCCAGCTCTTTA | 45 | 76.9° C. | 38.4° C. |
| 1 | GCCGCCGTCCATCTACACAAGCCCGGTCTATAAGTTACAT | 52 | 79.9° C. | 41.8° C. |
| 33 | CAACAACGAATGAGTATATTCAAACCTAGTGTTCGGTATG | 37 | 73.8° C. | 44.1° C. |
| 16 | AAGTCCACGACACTGCCTCCGCAAAACGTTGTTAAAAGTG | 47 | 77.9° C. | 44.2° C. |
| 45 | TTCCGAGAGACGTGAAAAATTTCAGAAATCAGCAACAACG | 40 | 74.8° C. | 44.9° C. |
| 8 | GCTCTTTAACTTAAAGTCGATTCGGGGCACTAACGGCTAA | 45 | 76.9° C. | 45.2° C. |
| 43 | TCTAGCCCGCTGCTGTCGGTTCCTTGGAATGCCCGCCGTC | 65 | 85.1° C. | 46.4° C. |
| 50 | TCGTATCCCCCACCGAAGCCAGCTTCCGCCGTCCAAGTAG | 62 | 84° C. | 47.0° C. |
| 34 | TAGTTGACCCCAAAGTTCCCCGATGACGACGTACATCTTC | 50 | 78.9° C. | 48.6° C. |
| 48 | TTCGAATAGTAAAGGAATACCATTATAGGTCGGTTATCGG | 37 | 73.8° C. | 48.7° C. |
| 6 | GGCCTACTCTCGTGGGTTCTACACTTAACTCACTACGCAT | 50 | 78.9° C. | 48.9° C. |
| 21 | TCGGTATGTAGTTGCCCCGAAATAACCCCTATGCCGCCGT | 55 | 81° C. | 51.2° C. |
| 37 | TAACCTTCCAAGGCATTTATTACTTATATCTGCATCATAC | 32 | 71.7° C. | 51.2° C. |
| 17 | TCGTCGTTCGAAGTAGAGACTCGGTGAGTATACTTTAGCA | 45 | 76.9° C. | 51.9° C. |
| 9 | GCCTCCGTCGCAGATGAGACTCGCTGAGATTACGATACCT | 55 | 81° C. | 52.1° C. |
| 40 | TTAGAAACCGAACGCTCGACCAGTTTTGGGACCTTTTAGC | 47 | 77.9° C. | 53.1° C. |
| 49 | ATTATTAAGTCCAACTCTCGAGTTATCAACACTAACCTTC | 35 | 72.8° C. | 54.7° C. |
| 24 | CTCACCCGTACCGTTCCAAGGGAAAAATTACTTAAATCTG | 42 | 75.8° C. | 55.2° C. |
| 28 | CTTTTAGCAGTAGTACGTGCTCCTGTGGAGTTAAGTCCAC | 47 | 77.9° C. | 55.5° C. |
| 35 | CTGGAGTGAACTCAACGTCACTGACTCCGCTAAAAGTTCG | 50 | 78.9° C. | 55.8° C. |
| 3 | GATTCTATCCGAGCCGTAGCGATCGAGGTGACCCCTAAGT | 55 | 81° C. | 56.3° C. |
| 42 | GCGTTGCCGCGAACTAACCGCTAAGCCTCCGGCGAAGGTG | 65 | 85.1° C. | 56.3° C. |
| 23 | AAAGTTGGGAAAACTGCTACTACCTAGGACGAGAGTCTAA | 42 | 75.8° C. | 56.8° C. |
| 13 | AAGGTATCGCCTTTCCCCCATAGATACCAGATGCAGCAGG | 52 | 79.9° C. | 58.0° C. |
| 22 | ACATCTTCACAAGACCGGACTTGTAGTGTCATTCTTATGG | 42 | 75.8° C. | 58.1° C. |
| 41 | GAAGAAGGACATCTTCTCAAGACAGGTCTGGTACTGTCAG | 47 | 77.9° C. | 60.0° C. |
| 30 | CGAAGGTGTGTCTCGCTCTGTTTACGTTACCTTGGAAAGG | 50 | 78.9° C. | 60.6° C. |
| 7 | TTTGCGAGCGTTGAGCATGATGAATTGCTCGTCTGGATTG | 47 | 77.9° C. | 60.7° C. |
| 12 | TCAAACAGGAACAACGAATTCGTCTCTACAAACCTCGTGT | 42 | 75.8° C. | 61.6° C. |
| 29 | ACTGTCAGTCTTATCGGGGACCTAAGGGGTCATCGTCGTT | 52 | 79.9° C. | 64.1° C. |
| 27 | CGCCTGGGTTCGACACCTAACGCACGACCATTTTAGGGCG | 60 | 83° C. | 64.4° C. |
| 4 | TAACAGTGCAAGAACCACTTCCGCGAGAGACAGAGCGGGT | 55 | 81° C. | 64.9° C. |
| 25 | CATCATACAATTCCGCGCGACAATCAGCGGGTAAGGTATC | 50 | 78.9° C. | 64.9° C. |
| 19 | ATAGTGGCTGATAAACGGAACGCTATTCCCTTTTTGCGAG | 45 | 76.9° C. | 65.0° C. |
| 39 | GGGCCCTGAGGGCTGATGGTGGTAGGCAGACGCGCCTGGG | 72 | 88.1° C. | 65.0° C. |
| 31 | CCGCCGTCCGCGCTCATGCTGCGTGGCCTTATATAGTGGC | 65 | 85.1° C. | 65.3° C. |
| 15 | TTAGGGCGGTTTTCGCATTATTACGATCACCTGTGGATTT | 42 | 75.8° C. | 65.7° C. |
| 47 | TTTGCGACCGTTGACCAGGAGGAAGTGCTCCTCTGGAGTG | 57 | 82° C. | 66.1° C. |
| 18 | TGGAAAGGGGGCCGTCCGGGCTCAAGCTGCTTGGCCTACT | 65 | 85.1° C. | 66.4° C. |
| 26 | ATTCCATGTGTGCGAGCGGTGAGCTTGATGTATTGCTCCT | 50 | 78.9° C. | 67.3° C. |
| 2 | ACTAATCGGGGCCCTAAGGGCTCATCGTCGTACGCAGAAG | 57 | 82° C. | 68.1° C. |
| 11 | GAGTCTAACAGACGCGTCCCGATGGACGTGCAAACTAAGA | 52 | 79.9° C. | 71.2° C. |
| 14 | TTGCTCCTCGCGATTGATCTCCCGGTCCGGGACTAGCCTA | 60 | 83° C. | 71.7° C. |
| 10 | TCTTATGOGGTACCACCGOGGACATGGTGGTTCTAATTAG | 50 | 78.9° C. | 73.3° C. |
| 32 | AAGCCGGGAACGGATCGCCTTGCACACATAGATAACAGAT | 50 | 78.9° C. | 80.9° C. |

Table I depicts random 40-mers with combined percent fraction of G and C nucleotides (% GC), predicted duplex melting temperature under PCR conditions (hybrid Tm), and predicted hairpin melting temperature (hairpin Tm). Sequences with lowest hairpin melting temperatures (highlighted) are selected for primer design.

Matching primers for PCR amplification (Tm ˜55° C.) are designed and potential tag oligonucleotide amplicons are ranked using the cycle crossing (Ct) difference between reaction containing and not containing the tag target. Oligonucleotide tags producing the greatest difference between reactions with and without target are selected for conjugation onto polymer.

Selected oligonucleotide tag sequence are synthesized by Integrated DNA Technologies (IDT) at 1 μmole or higher scale with a 3′ amino modifier attached as a selective reactive group for conjugation to polymer

### Example 2

### Removal of Residual Monomer from poly(4-vinylpyridine) Polymer Used for Purifying a Biomolecule of Interest

In a representative experiment, residual 4-vinyl pyridine monomer was removed from poly(4-vinylpyridine) prior to use in the methods described herein. Linear poly(4-vinylpyridine) (PVP) having molecular weight 200,000, obtained form SCIENTIFIC POLYMER PRODUCTS, INC., was spread evenly on a glass dish and placed in a vacuum oven. The atmosphere inside the oven was purged with argon several times for 5 minutes, in order to remove any oxygen. The pressure in the oven was reduced to 0.1 in mercury using a mechanical vacuum pump and subsequently the temperature was raised to about 120° C. The polymer was subjected to these conditions for a total of 24 hours. During this time, the atmosphere inside the oven was purged with argon several times for 5 minutes. At the end of the heating period, the oven temperature was lowered to room temperature and the oven was purged with argon several times before opening the door. The resulting polymer did not have a noticeable odor, whereas the untreated polymer has a distinct odor of 4-vinyl pyridine monomer. The amount of residual 4-vinyl pyridine monomer present in the treated polymer was not detectable by gel permeation chromatography whereas the untreated polymer had about 0.05% (w/w) residual 4-vinyl pyridine monomer.

### Example 3

### Modification of poly(4-vinyl pyridine) Polymer with DNA Tag Using Iodoacetamide Chemistry

Following the removal of the residual monomer from poly(4-vinylpyridine), the polymer was tagged with a DNA tag. In a representative experiment, a DNA tag comprising 40 nucleotides was used, having following sequence: 5′-GTT ATC GGATTC TTA ATA GGA ACA CAC TCT ATC TCA CCC G/3 AmM/-3′ (SEQ ID NO:1), also shown as ID 36 in Table I, and modified with a primary amine group.

Iodoacetamide (Ultragrade), 1,1′-carbonyldiimidazole (7%), acetonitrile, dimethylformamidem (DMF), and dimethyl sulfoxide (DMSO, anhydrous, ≧9.9%) were obtained from SIGMA. About 5.56 mg of iodoacetamide was activated with 4.48 mg of 1,1′-carbonyldiimidazole dissolved in 0.5 ml of anhydrous DMSO. The reaction was carried out at room temperature for about an hour, protected from light. About 3.71 mg of the DNA tag described above, was dissolved in 100 μA deionized (DI) water, followed by the drop wise addition of 0.4 ml DMSO. About 0.5 ml of the DNA tag solution was mixed with 0.5 ml of activated iodoacetamide solution and reacted at room temperature for 24 hours. The product, iodine terminated DNA tag, was precipitated using 0.5 ml acetonitrile, and collected as a pellet by centrifugation at 2500 rpm for 2 minutes. Excess, un-reacted iodoacetamide remained in the supernatant and the solution was discarded. The pellet was washed with 1 ml acetonitrile, and re-dissolved in 1.0 ml of DI water. The latter was added drop-wise to a solution of 50 mg PVP from Example 2, dissolved in 2 ml DMF. The mixture was reacted at a temperature of about 60° C. for 24 hours.

### Example 4

### Modification of poly(4-vinyl pyridine) Polymer with DNA Tag Using Epichlorohydrin

Following the removal of the residual monomer from poly(4-vinylpyridine), the polymer was tagged with an oligonucleotide DNA tag. In a representative experiment, a DNA tag comprising 40 nucleotides was used, having the following sequence: 5′-GTT ATC GGATTC TTA ATA GGA ACA CAC TCT ATC TCA CCC G/3AmM/-3′ (SEQ ID NO:1) and modified with a primary amine group.

Epichlorohydrin (99%), diethyl ether, dimethylformamidem (DMF), ethylacetate (ACS reagent grade), Methanol (ACS reagent grade), sodium tetraborate (99%) and Sodium Hydroxide (0.1N) were obtained from SIGMA. About 2.97 mg of the DNA tag described above, was dissolved in 1 ml sodium tetraborate solution (20 mM, pH 9.2), followed by the addition of 0.2 ml DMF containing 3 μl of Epichlorohydrin. The reaction was carried out at room temperature for about an hour. The solution was then extracted with 3 portions of 2 ml diethyl ether to remove excess un-reacted Epichlorohydrin. The purified solution was added drop-wise to a solution of 30 ml DMF containing 3 g of PVP from Example 2. The mixture was reacted at a temperature of about 50° C. for 24 hours.

### Example 5

### Removal of Excess DNA Tag from DNA Tagged poly(4-vinyl pyridine) Polymer

DNA tagged PVP from Example 4 was precipitated using equal volume of ethylacetate solution, redissolved in equal volume of methanol and finally precipitated using equal volume of 0.1M NaOH to remove unconjugated DNA tag. The precipitate was then dissolved in a mixture of DMF and water (50:50 volume ratio) to a final concentration of 6 wt %.

### Example 6

### Quantitation of DNA Tag Conjugated to poly(4-vinyl pyridine) Polymer

Following the removal of excess DNA tag, the amount of tag conjugated to the PVP was quantitated. Serial dilution standards of the unconjugated DNA tag were amplified using 12.5 μl Fast SYBR Green Master Mix (APPLIED BIOSYSTEMS), 0.9 μM of each primer with 2 μl of sample in a final volume of 25 μl using the amplification conditions of: one denaturation cycle of 95° C. for 60 seconds; 50 cycles of 95° C. for 2 seconds; and 50° C. for 30 seconds, followed by a final extension at 50° C. for 30 seconds. The amplification products were visualized by direct measurement of fluorescence to obtain a crossing threshold value (Ct).

A known volume of a known polymer concentration was amplified after 1:100 or more dilution and the Ct obtained was compared with that from the standard curve generated using unconjugated polymer to obtain the number of copies in the sample of conjugated polymer. This allowed a calculation of the number of DNA tag copies per molecule of poly(4-vinyl pyridine) polymer. For pilot batches, the conjugation ratio was determined to be roughly 1 DNA tag per 200 polymer molecules.

### Example 7

### pH Adjustment of an Unclarified Cell Culture Fluid

Cells derived from a non-expressing Chinese Hampster Ovary (CHO) cell line were grown in a bioreactor (NEW BRUNSWICK SCIENTIFIC) to a density of 10×10⁶ cells/ml in 10 L of culture medium and harvested at 64% viability. IgG was spiked to a concentration of 1.0 g/L and the concentration of host cell proteins (HCP) was determined to be 385669 ng/ml. The pH of the fluid was 7.2. The pH of the unclarified cell culture fluid was adjusted to 4.5 using 0.17 ml of 3.0M acetic acid, prior to the start of the purification process.

### Example 8

### Preparation of Purified DNA Tagged PVP Solution in Acetic Acid

The solution from Example 5 was dried in a vacuum oven operated at a temperature of 60° C. for 24 hr. The remaining solid was reconstituted in 3M acetic acid, with continuous agitation for 1 hour at room temperature, to a final concentration of 15 wt %.

### Example 9

### Use of Oligonucleotide Tag-Conjugated Polymer for Downstream Process Monitoring

0.5 ml of PVP solution from Example 8 and 0.56 g ammonium sulfate were added to 10 ml of un-clarified cell culture fluid from Example 7 and mixed at room temperature for 5 minutes in order to allow for binding of insoluble impurities, such as cells and cell debris as well as soluble impurities, such as host cell proteins, nucleic acids, etc. The polymer-impurities complex, was then precipitated by adjusting the pH of the mixture to 8.5 using 1.45 ml of 2M Tris containing 0.08 g ammonium sulfate, this allows the desired product (IgG) to hind to the complex. The precipitate, in the form of a dispersed solid suspension, was mixed continuously in the fluid for 10 min. The precipitate containing the IgG complex was subsequently collected by centrifugation (4000 rpm for 1 min) and washed with 1M ammonium sulfate (10 mM Tris, pH 8.5) to remove loosely-bound impurities. While cells, cell debris and other soluble impurities remained bound to the precipitate, IgG was selectively eluted from the precipitate at pH 5.3 (100 mM sodium acetate), while mixing for 20 mins, followed by filtration through 0.2 μm Durapore® filters. Under these conditions, 93% of the IgG present in the original sample was bound to the polymer and the percentage of IgG recovered after elution was 80 wt %.

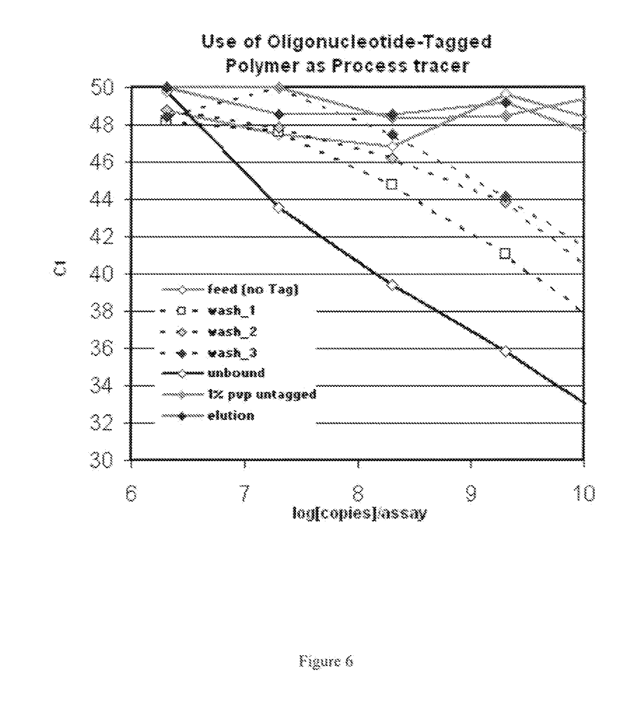

Residual PVP in the different steps of the purification process was quantified as described in Example 6 and in FIG. 6. As the fractions move through the purification process, the curves begin to flatten out with a crossing threshold (Ct) of around 48 for all dilutions, similar to the no tag control.

### Example 10

### Preparation of Unclarified Non-Expressing Cell Culture Fluid (CCF)

In a representative experiment, cells derived from a Chinese Hamster Ovary (CHO) cell line expressing a monoclonal IgG₁ were grown in a 10 L bioreactor (NEW BRUNSWICK SCIENTIFIC) to a density of 13×10⁶ cells/mL and harvested at <50% viability. The antibody titer was determined to be 0.7 mg/mL via protein A HPLC. The level of host cell proteins (HCP) was found to be 141800 ng/mL using an ELISA (CYGNUS # F550) and the DNA concentration was determined to be 11.9 ug/mL by PicoGreen® assay. The pH of the unclarified cell culture was pH 7.2.

### Example 11

### Preparation of a Hydrophobically Modified Polyallylamine Based Stimuli Responsive Polymer

In a representative experiment described herein, it was demonstrated that the polymers described herein could be readily manufactured on a large scale.

Using a solution of polyallylamine (PAA, NITTOBO, 150 kD; 40% wt./wt.), a hydrophobically modified stimulus responsive polymer was produced as follows.

500 g of Polyallylamine (PAA, NITTOBO, 150 kD; 40% wt./wt.) (approximately 200 g of polyallylamine) was added to a 4 liter glass jar. Next, 80 g of NaOH pellets and 1000 mL of deionized water were dissolved and added to the jar. This was followed by the addition of 1000 mL 1,2-dimethoxyethane (SIGMA) as a co-solvent and the solution was stirred vigorously until it was homogenous. Next, 114 g of benzyl chloride (ACROS ORGANICS, 99%) was added to the reaction jar. The solution was heated at 60° C. for 16 hours with magnetic stirring. The solution was allowed to cool to room temperature and transferred to a 10 liter beaker.

Next, 1000 mL of deionized water was added with stirring and a sticky solid mass precipitated out of solution. The product was further precipitated with slow addition of 200 mL of 2 M sodium phosphate and the solid was collected and washed with deionized water. The polymer was further purified by the following method.

The solid was dissolved in 3 liters of 1 M acetic acid with stirring. The total volume was brought to 10 liters with deionized water and the pH was adjusted to 7 with drop wise addition of 50% NaOH. The product was precipitated with the addition of 800 g of 2 M sodium phosphate and the solids was collected and washed with deionized water. The polymer was even further purified by the following method. The solid was dissolved in 3 liters of 1 M acetic acid with stirring. The total volume was brought to 10 liters with deionized water and the pH was adjusted to 7.0 with drop wise addition of 50% NaOH. The product was precipitated with addition 800 g of 2 M sodium phosphate and the solids was collected and washed with deionized water.

The resulting solid mass was dried in a vacuum oven at 65° C. for 3 days. The dried polymer was frozen with liquid nitrogen and ground to a fine powder and further dried for 1 day. The resulting mass of the dry powder was 250 g. A small sample was dissolved in 1 M CD₃COOD/D₂O acid and ¹H-NMR spectra was obtained. The ¹H-NMR peaks were integrated and the amount of benzyl modification was determined to be 33%.

The resulting powder was dissolved to make a 10% w/w solution in 1 M acetic acid. The resulting solution was tested for sensitivity to a multivalent ion stimulus by drop wise addition of 2 M sodium phosphate or 0.2 M sodium citrate to 5 mL samples of 0.5% polymer solutions. Upon addition of the phosphate or citrate ions, a white precipitate is observed, thereby indicating that the polymer was responsive to a multivalent anion stimulus.

### Example 12

### Labeling of a Hydrophobically Modified Polyallylamine Stimulus Responsive Polymer with a Fluorescence Tag

The resulting polymer from Example 11 was labeled with a fluorescent tag by the following method. 2 g of the solid polymer was added to 10 mL of 1 M acetic acid and stirred until the solid is dissolved. The resulting solution volume was increased to 100 mL by addition of deionized water. The pH of the solution was adjusted to 8.5 with addition of 4 M NaOH. A reactive dye solution is prepared by adding 12 mg of N-(4,4-difluoro-1,3,5,7-tetramethyl-4-bora-3a,4a-diaza-s-indacene-2-yl)iodoacetamide (BODIPY® 507/545 IA) (INVITROGEN) to 6 mL DMF. Immediately after preparation, the reactive dye solution was added to the polymer solution with vigorous stirring. The reaction solution was rotated at 50° C. for 1 hour. Next, the solution was centrifuged and the supernatant was decanted and discarded. The red colored pellet was resuspended in 50 mM potassium phosphate pH 7 and agitated. The solution was subsequently centrifuged and the supernatant was decanted and discarded.

The red colored solid polymer was further purified by dissolving in 10 mL of 1 M acetic acid. The resulting orange/red solution volume was increased to 500 mL by addition of deionized water. The pH was adjusted to 7 and the solution was brought to a concentration of 50 mM potassium phosphate by drop wise addition of 2 M potassium phosphate and a red precipitate was observed. The purification was repeated one more time and a bright red solid polymer was recovered and washed with deionized water. The red solid was dried under vacuum at 50° C. for 2 days. Finally, 1.27 g of dried red polymer was ground to a fine powder and dissolved in 1 M acetic acid to make a 5% w/w solution.

### Example 13

### Detection of Residual Amounts of Stimulus Responsive Polymer with a Fluorescence Tag

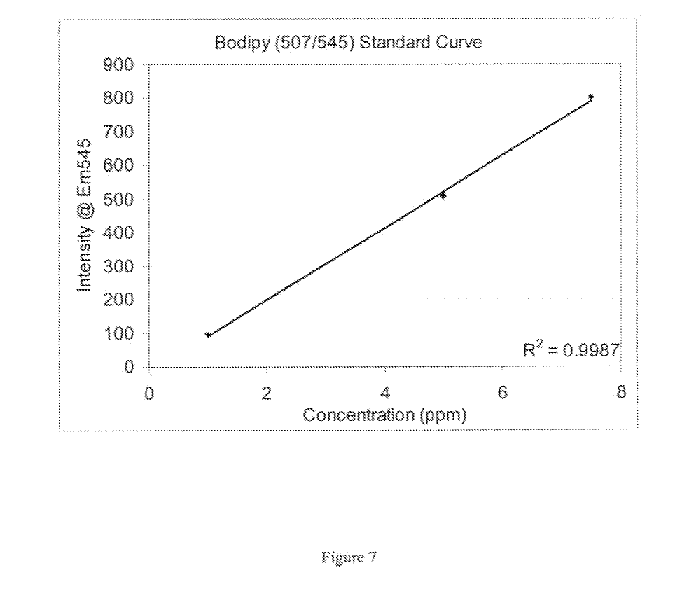

Using a 5% solution of BODIPY 507/545 tagged, hydrophobically modified polyallylamine from Example 12, standard solution concentrations of 1000, 100, 10, 1, and 0.1 ppm solids were prepared in deionized water. A fluorescence spectrophotometer was set at excitation of 507 nm and emission of 545 nm. The intensity at 545 nm was recorded for each sample and a standard curve was plotted, as depicted in FIG. 7.

In order to determine the efficiency of polymer precipitation using a multivalent anion stimulus as well as the concentration of residual polymer, the following experiment was performed.

In 5 mL deionized water, 4000 ppm of the tagged polymer was spiked. The pH was adjusted to 7 and the concentration of potassium phosphate was brought to 50 mM. At this time, a red precipitate separates and the vial was centrifuged at 3000 rpm for 2 minutes. The turbidity of the resulting supernatant was determined to be 46 NTU and the residual polymer in the supernatant was determined to be 44 ppm by fluorescence spectroscopy. The supernatant was filtered through a 0.22 micron Durapore Millex (MILLIPORE, 33 mm) and the residual polymer in the filtrate was determined to be 30 ppm by fluorescence spectroscopy. Notably, this experiment resulted in 99.25% removal of high levels of residual polymer in water with simple addition of a stimulus, centrifugation, and filtration.

These results demonstrate that a stimulus responsive polymer comprising of a fluorescence tag can be added to water, rendered insoluble by addition of a stimulus, the solids can be separated and the residual polymer can be detected by fluorescence spectroscopy.

### Example 14

### Determination of Residual Amount of Stimulus Responsive Polymer with a Fluorescence Tag in a Flocculated CHO Cell Culture

Using a 5% solution of BODIPY 507/545 tagged, hydrophobically modified polyallylamine from Example 12, standard solution concentrations of 1000, 100, 10, 1, and 0.1 ppm solids were prepared in deionized water. A fluorescence spectrophotometer was set with excitation at 507 nm and emission at 545 nm. The intensity at 545 nm was recorded for each sample and a standard curve was plotted according to Example 13.

CHO cell culture was prepared using a method similar to Example 10. Residual polymer from flocculation and precipitation of the cell culture using a catonic smart polymer subjected to a stimulus was compared to a using a catonic smart polymer with no stimulus. Dose of 0.1, 0.4 or 0.6 w/v of tagged polymer was added to 5 mL of unclarified CHO cell culture in duplicates in 15 mL conical tubes.

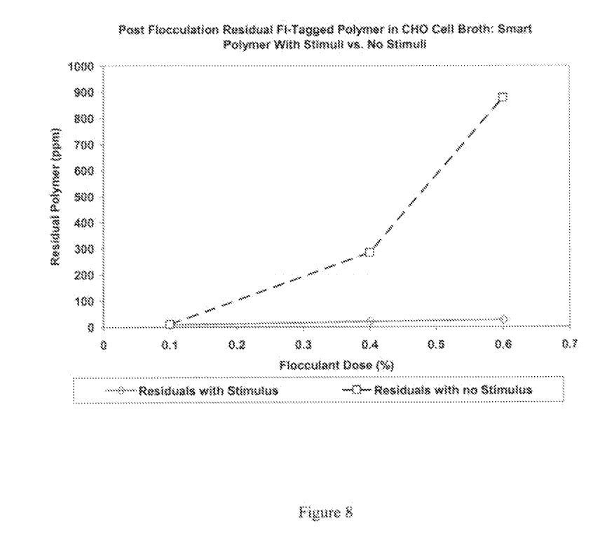

One of the series of the three polymer concentrations was adjusted to pH 7.2 with 2 M tris base, the potassium phosphate concentration was increased to 50 mM by drop wise addition of 2 M potassium phosphate and the solution was stirred. The sodium phosphate and pH adjustment was performed in order to precipitate the stimulus responsive polymer along with a complex of cells, cell debris, impurities, residual polymer as well as to flocculate the solids and increase particle size. The second series of three polymer concentration are simply stirred and no stimulus was applied. The vial was centrifuged at 3000 rpm for 2 minutes. The turbidity of the resulting supernatant was recorded and the supernatants were filtered through a 0.22 micron Durapore Millex (MILLIPORE, 33 mm). The residual polymer in the filtrate was determined by fluorescence spectroscopy. A CHO cell culture was used as control and is subtracted as a blank. The results are tabulated in Table II and depicted in FIG. 8.

As shown in Table H and in FIG. 8, these results demonstrate that a stimulus responsive polymer comprising of a fluorescence tag can be added to water, rendered insoluble by addition of a stimulus, the solids can be separated and the residual polymer can be detected by fluorescence spectroscopy. The results also demonstrate the advantage of control over residual polymer by applying a stimulus to a stimulus responsive flocculent or precipitant in cell culture.

TABLE II

| Intial | | | stimuli | |
|---|---|---|---|---|
| polymer | Initial | turbidiy of | applied (yes/ | residual polymer in |
| spike (%) | (ppm) | centrate (NTU) | no) | filtrate (ppm) |
| 0 | 0 | 237 | no | 0.0 |
| 0.1 | 1000 | 11 | yes | 9.8 |
| 0.4 | 4000 | 67 | yes | 21.1 |
| 0.6 | 6000 | 54 | yes | 27.1 |
| 0.1 | 1000 | 9 | no | 11.3 |
| 0.4 | 4000 | >1000 | no | 283.7 |
| 0.6 | 6000 | >1000 | no | 876.7 |

### Example 15

### Detection of a Stimulus Responsive Polymer Tagged with a Fluorescence Tag Following Use with a Protein a Capture of a Monoclonal Antibody from a Clarified Cell Culture

Using a 5% solution of BODIPY 507/545 tagged, hydrophobically modified polyallylamine from Example 12, standard solution concentrations of 1000, 100, 10, 1, and 0.1 ppm solids is prepared in deionized water. A fluorescence spectrophotometer was set with excitation of 507 and emission of 545. The intensity at 545 nm is recorded for each sample and a standard curve was plotted according to Example 13.

CHO cell culture was prepared using a method similar to Example 10. The cell culture was clarified by flocculation using a dose of 0.4% w/w BODIPY 507/545 tagged, hydrophobically modified polyallylamine from Example 12. The pH of the cell culture was adjusted to 7.0 by drop wise addition of 2 M tris base. Next, 2 M potassium phosphate was added so that the final potassium phosphate concentration was 50 mM. At this point there was an aggregation of solids in the solution. The solution was centrifuged and the pellet was discarded. The supernatant was filtered through a 0.22 micron Durapore filter (MILLIPORE) and the filtrate was collected and pooled.

1 mL of Protein A affinity chromatography resin (ProSep Ultra Plus®) was packed in a Omnifit chromatography column with a 0.66 cm internal diameter (DIBA INDUSTRIES) to a final bed height of 2.9 cm. The protein A column was loaded to a density of 35 mg/mL IgG at 3 minute residence time and the flow through was collected. The column was subsequently washed with 50 mM tris pH 7.2 and the wash pool was collected. Next, the IgG was eluted with 5 column volumes with 50 mM acetic acid and the elution pool was collected. Absorbance at 280 nm was recorded for the elution pool and from this absorbance, the yield was determined to be 97 percent via mass balance. Finally, the column was regenerated with 0.15 M phosphoric acid and the pool was collected. The samples were neutralized to pH 7.0 with 2 M iris base.

A fluorescence spectrophotometer was set with excitation at 507 nm and emission at 545 nm. The intensity at 545 nm is recorded for each sample. The residual polymer was quantified for the cell culture supernatant as well as for each pool collected from the Protein A chromatography column, as shown in Table III. The limit of quantification for the assay was determined to be 1.0 ppm.

The results demonstrate that a fluorescently tagged smart polymer can be detected in a cell culture supernatant and the residual polymer removal can be traced during downstream purification of the target molecule.

TABLE III

| Step | Residual Polymer (ppm) |
|---|---|
| Filtrate | 12.3 |
| Protein A Flow Through | 11.4 |
| Protein A Intermediate Wash | 0.6 (LOQ) |
| Protein A Elution | 0.2 (LOQ) |
| Protein A Regeneration | 0.2 (LOQ) |

*LOQ = Limit of Quantification
*Initial Spike = 0.4 percent w/w or 4000 ppm

### Example 16

### Biotinylation of a Stimulus Responsive Polymer

A stimuli responsive polymer based on a hydrophobically modified polyallylamine was synthesized Similar to Example 11. The resulting polymer was labeled with a biotin tag using the following method.

20 g of a 5% w/w solution of a 33% benzylated polyallylamine with a molecular weight of 150,000 Da was added to a 1000 mL glass beaker equipped with a magnetic stirrer. The volume was increased to 200 mL by the addition of deionized water and the pH was adjusted to 9.5 by the drop wise addition of 4 M NaOH.

A reactive biotin solution was prepared by dissolving 15 mg of DSB-X™ biotin C₂-iodoacetamide (desthiobiotin-X C₂-iodoacetamide) (INVITROGEN) in 6 mL of Dimethylformamide (DMF). The reactive biotin solution was immediately added to the glass beaker containing the pH adjusted 33% benzylated polyallylamine solution. The resulting mixture was stirred vigorously, heated to 70° C. and maintained at these conditions for 1 hour. Next, the mixture was allowed to cool to room temperature and was diluted to 1000 mL with deionized water. The biotinylated polymer was isolated via precipitation with a drop wise addition of 2 M sodium phosphate until no further precipitate was observed. The solution was vacuum filter through a nonwoven filter material and the solid was collected while the filtrate was discarded. The solid polymer product was washed 100 mL of deionized water.

The biotinylated product was further purified by dissolving the solid mass in 50 ml of 1 M acetic acid over night. The solution was brought to a volume of 2 liters and the pH was adjusted to 7.0 by a drop wise addition of 4 M NaOH. The biotinylated product was isolated via precipitation with a drop wise addition of 2 M sodium phosphate until no further precipitate was observed. The solution was vacuum filter through a nonwoven filter material and the solid was collected while the filtrate was discarded. The solid was washed 100 mL of deionized water and placed in a vacuum oven at 60° C. overnight.

The resulting dried biotinylated product was ground to a powder and dissolved in 1 M acetic acid and 0.05 molar HCl to a final concentration 5% w/w.

It is understood that the resulting biotinylated smart polymer could be used to flocculate or purify a target molecule from a cell culture or protein containing feed. Once the polymer is added to the cell culture or protein containing feed and a stimulus is applied, the solid mass could be separated from the supernatant by centrifugation, filtration, settling, any other method of solid liquid separation, or any combination of these methods. Furthermore, the clarified fluid could be passed through a filter specifically designed for removal of residual polymer before or after quantification.

Finally, any residual biotinylated smart polymer could be detected and quantified via existing methods for detecting and quantifying biotinylated molecules. The detection and quantification methods may include avidin/streptavidin-tagged detection strategies, including strategies involving enzyme reporters (e.g., horseradish peroxidase, alkaline phosphatase) or fluorescent probes. The detection and quantification could also be performed with an immunoassay such as an ELISA for example.

An example of a detection kit includes the Fluorescence Biotin Quantitation Kit available from THERMO SCIENTIFIC. Yet another example of a detection kit that could be used is the Biotin ELISA Kit from ALPCO DIAGNOSTICS.

The specification is most thoroughly understood in light of the teachings of the references cited within the specification which are hereby incorporated by reference. The embodiments within the specification provide an illustration of embodiments in this invention and should not be construed to limit its scope. The skilled artisan readily recognizes that many other embodiments are encompassed by this invention. All publications and inventions are incorporated by reference in their entirety. To the extent that the material incorporated by reference contradicts or is inconsistent with the present specification, the present specification will supercede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

What is claimed is:

1. A method of detecting residual amounts of a polymer in a sample comprising a biomolecule of interest, wherein the polymer is used for separating the biomolecule of interest from one or more impurities, the method comprising the steps of: (1) contacting the sample with the polymer, wherein the polymer is associated with a tag; and (2) detecting the tag,
wherein the amount of tag detected is indicative of the amount of residual polymer in the solution comprising the biomolecule of interest.

2. The method of claim 1, wherein the polymer is associated with an oligonucleotide tag.

3. The method of claim 2, wherein the oligonucleotide tag is detected using
an amplification reaction.

4. The method of claim 3, wherein the amplification reaction comprises addition of a set of primers capable of hybridizing to the 5′ and 3′ ends of the oligonucleotide tag.

5. The method of claim 2, wherein the oligonucleotide tag is associated with a hapten molecule, a fluorescent molecule or a radioactive molecule.

6. The method of claim 2, wherein the detecting step comprises a non-amplification reaction.

7. The method of claim 6, wherein the non-amplification reaction comprises
use of a labeled probe.

8. The method of claim 7, wherein the labeled probe comprises a detectable
dye.

9. The method of claim 1, wherein the polymer is associated with the oligonucleotide tag using covalent attachment.

10. The method of claim 3, wherein the amplification reaction comprises polymerase chain reaction.

11. The method of claim 2, wherein the oligonucleotide tag comprises a length selected from the group consisting of 10 nucleotides, 15, nucleotides, 20 nucleotides, 25 nucleotides, 30 nucleotides, 35 nucleotides, 40 nucleotides, 45 nucleotides, 50 nucleotides, 55 nucleotides and 60 nucleotides.

12. The method of claim 2, wherein the oligonucleotide tag comprises a length of 60 nucleotides or less than 60 nucleotides.

13. The method of claim 11, wherein the oligonucleotide tag comprises a reactive group at the 3′ end of the tag.

14. The method of claim 12, wherein the oligonucleotide tag comprises a reactive group at the 3′ end of the tag.

15. The method of claim 13, wherein the reactive group is selected from a group consisting of a halogen group, an epoxy group, hydroxyl group, an amino group, a sulfhydryl group and carboxyl group.

16. The method of claim 14, wherein the reactive group is selected from a group consisting of a halogen group, an epoxy group, hydroxyl group, an amino group, a sulfhydryl group and carboxyl group.

17. The method of claim 15, wherein the reactive group is further modified.

18. The method of claim 16, wherein the reactive group is further modified.

19. The method of claim 1, wherein the polymer comprises a reactive group.

20. The method of claim 1, wherein oligonucleotide tag comprises a first reactive group and the polymer comprises a second reactive group, and wherein the first and the second reactive groups are covalently attached to each other.

21. The method of claim 1, wherein the oligonucleotide tag not associated with the polymer is removed by purification.

22. The method of claim 1, wherein the biomolecule is selected from the group consisting of a protein, an antibody and a vaccine.

23. The method of claim 22, wherein the antibody is a monoclonal antibody.

24. The method of claim 1, wherein the oligonucleotide tag comprises the sequence set forth in SEQ ID NO:1.

25. The method of claim 1, wherein the oligonucleotide tag comprises a nucleotide sequence predicted to be free of secondary structure.

26. The method of claim 1, wherein the oligonucleotide tag comprises a nucleotide sequence free of homopolymers of four or more bases in length.

27. The method of claim 1, wherein the polymer is poly(4-vinyl pyridine).

28. The method of claim 1, wherein the polymer is polyallylamine.

29. The method of claim 1, wherein the polymer is polyvinylamine

30. The method of claim 1, wherein the polymer is a stimulus responsive polymer.

31. The method of claim 30, wherein the stimulus responsive polymer is responsive to a salt stimulus.

32. The method of claim 30, wherein the stimulus responsive polymer is responsive to a pH stimulus.

33. The method of claim 1, wherein the polymer contains primary amines and a hydrophobic portion.

34. The method of claim 1, wherein the polymer is polyvinylamine with 1-80% of its amines covalently modified with an aromatic group.

35. The method of claim 30, wherein the polymer is responsive to a multivalent anion Stimulus.

36. The method of claim 1, wherein the detecting step comprises an Immunoassay or ELISA

37. The method of claim 1, wherein the polymer separates the biomolecule of interest from one or more impurities by binding and precipitating one or more impurities in a sample comprising the biomolecule of interest and one or more impurities.

38. The method of claim 1, wherein the polymer separates the biomolecule of interest from one or more impurities by binding and precipitating the biomolecule of interest in a sample containing the biomolecule of interest and one or more impurities.

39. The method of claim 1, wherein the polymer is associated with a fluorescent tag or a radioactive tag or a hapten tag.

40. A method for detecting residual amounts of a stimulus responsive polymer used for separating a biomolecule of interest from one or more impurities, the method comprising the steps of:
(1) contacting a solution containing a biomolecule of interest and one more impurities with a stimulus responsive polymer, where the polymer is associated with a tag, such that to form a complex of polymer and one or more impurities;
(2) applying a stimulus to the solution, thereby to precipitate the complex;
(3) removing the precipitate from the solution; and
(4) detecting the tag in the solution containing the biomolecule of interest, where the amount of tag detected is indicative of the amount of residual polymer in the solution comprising the biomolecule of interest.

41. The method of claim 40, wherein the stimulus responsive polymer contains primary amines and a hydrophobic portion.

42. The method of claim 40 wherein the stimulus responsive polymer is associated with a fluorescent tag or a radioactive tag or a hapten tag.

43. The method of claim 40, wherein the stimulus responsive polymer is responsive to a multivalent anion stimulus.

44. The method of claim 42, wherein the hapten tag comprises biotin.

45. The method of claim 40, wherein the detecting step comprises use of an ELISA assay.

46. A method for detecting residual amounts of a stimulus responsive polymer used for separating a biomolecule of interest from one or more impurities in a solution, the method comprising the steps of:
(1) contacting a solution containing a biomolecule of interest and one or more impurities with a stimulus responsive polymer, where the polymer is associated with a tag and where the polymer forms complexes with both the biomolecule of interest as well as the one or more impurities under a first set of conditions;
(2) adding a stimulus to the solution, thereby to precipitate the complexes;
(3) subjecting the precipitate to a second set of conditions, thereby to selectively elute the biomolecule of interest from the complex; and
(4) detecting the tag in the eluate containing the biomolecule of interest.
wherein the amount of tag detected is indicative of the amount of residual polymer in the eluate.

47. The method of claim 46, wherein the method further comprises a washing step between steps (2) and (3).


SEQUENCE LISTING (50 sequences)

SEQ ID NO: 1 - DNA, 40 bp, Artificial Sequence
  Description of Artificial Sequence Synthetic oligonucleotide
gttatcggattcttaataggaacacactctatctcacccg

SEQ ID NO: 2 - DNA, 40 bp, Artificial Sequence
  Description of Artificial Sequence Synthetic oligonucleotide
ccaattagagagggccggattaaaggtaagctattccatg

SEQ ID NO: 3 - DNA, 40 bp, Artificial Sequence
  Description of Artificial Sequence Synthetic oligonucleotide
aatgagtagattcaatccgagggttaggtatctagttgac

SEQ ID NO: 4 - DNA, 40 bp, Artificial Sequence
  Description of Artificial Sequence Synthetic oligonucleotide
tatttctccataatacttggccgagagtaattaagccggg

SEQ ID NO: 5 - DNA, 40 bp, Artificial Sequence
  Description of Artificial Sequence Synthetic oligonucleotide
ctttagcaagtacatgggtcggaggtgggagaatgttgta

SEQ ID NO: 6 - DNA, 40 bp, Artificial Sequence
  Description of Artificial Sequence Synthetic oligonucleotide
taacagatccagcacgccatcttctatagccagctcttta

SEQ ID NO: 7 - DNA, 40 bp, Artificial Sequence
  Description of Artificial Sequence Synthetic oligonucleotide
gccgccgtccatctacacaagcccggtctataagttacat

SEQ ID NO: 8 - DNA, 40 bp, Artificial Sequence
  Description of Artificial Sequence Synthetic oligonucleotide
caacaacgaatgagtatattcaaacctagtgttcggtatg

SEQ ID NO: 9 - DNA, 40 bp, Artificial Sequence
  Description of Artificial Sequence Synthetic oligonucleotide
aagtccacgacactgcctccgcaaaacgttgttaaaagtg

SEQ ID NO: 10 - DNA, 40 bp, Artificial Sequence
  Description of Artificial Sequence Synthetic oligonucleotide
ttccgagagacgtgaaaaatttcagaaatcagcaacaacg

SEQ ID NO: 11 - DNA, 40 bp, Artificial Sequence
  Description of Artificial Sequence Synthetic oligonucleotide
gctctttaacttaaagtcgattcggggcactaacggctaa

SEQ ID NO: 12 - DNA, 40 bp, Artificial Sequence
  Description of Artificial Sequence Synthetic oligonucleotide
tctagcccgctggtgtcggttccttggaatgcccgccgtc

SEQ ID NO: 13 - DNA, 40 bp, Artificial Sequence
  Description of Artificial Sequence Synthetic oligonucleotide
tcgtatcccccaccgaagccagcttccgccgtccaagtag

SEQ ID NO: 14 - DNA, 40 bp, Artificial Sequence
  Description of Artificial Sequence Synthetic oligonucleotide
tagttgaccccaaagttccccgatgacgacgtacatcttc

SEQ ID NO: 15 - DNA, 40 bp, Artificial Sequence
  Description of Artificial Sequence Synthetic oligonucleotide
ttcgaatagtaaaggaataccattataggtcggttatcgg

SEQ ID NO: 16 - DNA, 40 bp, Artificial Sequence
  Description of Artificial Sequence Synthetic oligonucleotide
ggcctactctcgtgggttctacacttaactcactacgcat

SEQ ID NO: 17 - DNA, 40 bp, Artificial Sequence
  Description of Artificial Sequence Synthetic oligonucleotide
tcggtatgtagttgccccgaaataacccctatgccgccgt

SEQ ID NO: 18 - DNA, 40 bp, Artificial Sequence
  Description of Artificial Sequence Synthetic oligonucleotide
taaccttccaaggcatttattacttatatctgcatcatac

SEQ ID NO: 19 - DNA, 40 bp, Artificial Sequence
  Description of Artificial Sequence Synthetic oligonucleotide
tcgtcgttcgaagtagagactcggtgagtatactttagca

SEQ ID NO: 20 - DNA, 40 bp, Artificial Sequence
  Description of Artificial Sequence Synthetic oligonucleotide
gcctccgtcgcagatgagactcgctgagattacgatacct

SEQ ID NO: 21 - DNA, 40 bp, Artificial Sequence
  Description of Artificial Sequence Synthetic oligonucleotide
ttagaaaccgaacgctcgaccagttttgggaccttttagc

SEQ ID NO: 22 - DNA, 40 bp, Artificial Sequence
  Description of Artificial Sequence Synthetic oligonucleotide
attattaagtccaactctcgagttatcaacactaaccttc

SEQ ID NO: 23 - DNA, 40 bp, Artificial Sequence
  Description of Artificial Sequence Synthetic oligonucleotide
ctcacccgtaccgttccaagggaaaaattacttaaatctg

SEQ ID NO: 24 - DNA, 40 bp, Artificial Sequence
  Description of Artificial Sequence Synthetic oligonucleotide
cttttagcagtagtacgtgctcctgtggagttaagtccac

SEQ ID NO: 25 - DNA, 40 bp, Artificial Sequence
  Description of Artificial Sequence Synthetic oligonucleotide
ctggagtgaactcaacgtcactgactccgctaaaagttcg

SEQ ID NO: 26 - DNA, 40 bp, Artificial Sequence
  Description of Artificial Sequence Synthetic oligonucleotide
gattctatccgagccgtagcgatcgaggtgacccctaagt

SEQ ID NO: 27 - DNA, 40 bp, Artificial Sequence
  Description of Artificial Sequence Synthetic oligonucleotide
gcgttgccgcgaactaaccgctaagcctccggcgaaggtg

SEQ ID NO: 28 - DNA, 40 bp, Artificial Sequence
  Description of Artificial Sequence Synthetic oligonucleotide
aaagttgggaaaactgctactacctaggacgagagtctaa

SEQ ID NO: 29 - DNA, 40 bp, Artificial Sequence
  Description of Artificial Sequence Synthetic oligonucleotide
aaggtatcgcctttcccccatagataccagatgcagcagg

SEQ ID NO: 30 - DNA, 40 bp, Artificial Sequence
  Description of Artificial Sequence Synthetic oligonucleotide
acatcttcacaagaccggacttgtagtgtcattcttatgg

SEQ ID NO: 31 - DNA, 40 bp, Artificial Sequence
  Description of Artificial Sequence Synthetic oligonucleotide
gaagaaggacatcttctcaagacaggtctggtactgtcag

SEQ ID NO: 32 - DNA, 40 bp, Artificial Sequence
  Description of Artificial Sequence Synthetic oligonucleotide
cgaaggtgtgtctcgctctgtttacgttaccttggaaagg

SEQ ID NO: 33 - DNA, 40 bp, Artificial Sequence
  Description of Artificial Sequence Synthetic oligonucleotide
tttgcgagcgttgagcatgatgaattgctcgtctggattg

SEQ ID NO: 34 - DNA, 40 bp, Artificial Sequence
  Description of Artificial Sequence Synthetic oligonucleotide
tcaaacaggaacaacgaattcgtctctacaaacctcgtgt

SEQ ID NO: 35 - DNA, 40 bp, Artificial Sequence
  Description of Artificial Sequence Synthetic oligonucleotide
actgtcagtcttatcggggacctaaggggtcatcgtcgtt

SEQ ID NO: 36 - DNA, 40 bp, Artificial Sequence
  Description of Artificial Sequence Synthetic oligonucleotide
cgcctgggttcgacacctaacgcacgaccattttagggcg

SEQ ID NO: 37 - DNA, 40 bp, Artificial Sequence
  Description of Artificial Sequence Synthetic oligonucleotide
taacagtgcaagaaccacttccgcgagagacagagcgggt

SEQ ID NO: 38 - DNA, 40 bp, Artificial Sequence
  Description of Artificial Sequence Synthetic oligonucleotide
catcatacaattccgcgcgacaatcagcgggtaaggtatc

SEQ ID NO: 39 - DNA, 40 bp, Artificial Sequence
  Description of Artificial Sequence Synthetic oligonucleotide
atagtggctgataaacggaacgctattccctttttgcgag

SEQ ID NO: 40 - DNA, 40 bp, Artificial Sequence
  Description of Artificial Sequence Synthetic oligonucleotide
gggccctgagggctgatggtggtaggcagacgcgcctggg

SEQ ID NO: 41 - DNA, 40 bp, Artificial Sequence
  Description of Artificial Sequence Synthetic oligonucleotide
ccgccgtccgcgctcatgctgcgtggccttatatagtggc

SEQ ID NO: 42 - DNA, 40 bp, Artificial Sequence
  Description of Artificial Sequence Synthetic oligonucleotide
ttagggcggttttcgcattattacgatcacctgtggattt

SEQ ID NO: 43 - DNA, 40 bp, Artificial Sequence
  Description of Artificial Sequence Synthetic oligonucleotide
tttgcgaccgttgaccaggaggaagtgctcctctggagtg

SEQ ID NO: 44 - DNA, 40 bp, Artificial Sequence
  Description of Artificial Sequence Synthetic oligonucleotide
tggaaagggggccgtccgggctcaagctgcttggcctact

SEQ ID NO: 45 - DNA, 40 bp, Artificial Sequence
  Description of Artificial Sequence Synthetic oligonucleotide
attccatgtgtgcgagcggtgagcttgatgtattgctcct

SEQ ID NO: 46 - DNA, 40 bp, Artificial Sequence
  Description of Artificial Sequence Synthetic oligonucleotide
actaatcggggccctaagggctcatcgtcgtacgcagaag

SEQ ID NO: 47 - DNA, 40 bp, Artificial Sequence
  Description of Artificial Sequence Synthetic oligonucleotide
gagtctaacagacgcgtcccgatggacgtgcaaactaaga

SEQ ID NO: 48 - DNA, 40 bp, Artificial Sequence
  Description of Artificial Sequence Synthetic oligonucleotide
ttgctcctcgcgattgatctcccggtccgggactagccta

SEQ ID NO: 49 - DNA, 40 bp, Artificial Sequence
  Description of Artificial Sequence Synthetic oligonucleotide
tcttatggggtaccaccggggacatggtggttctaattag

SEQ ID NO: 50 - DNA, 40 bp, Artificial Sequence
  Description of Artificial Sequence Synthetic oligonucleotide
aagccgggaacggatcgccttgcacacatagataacagat